(19) United States
(12) Patent Application Publication
(10) Pub. No.: US 2014/0155767 A1
(43) Pub. Date: Jun. 5, 2014

(54) BIOLOGICAL INFORMATION MEASUREMENT APPARATUS, BIOLOGICAL INFORMATION MEASUREMENT SYSTEM, BIOLOGICAL INFORMATION MEASUREMENT METHOD, AND PROGRAM

(71) Applicant: Sony Corporation, Tokyo (JP)

(72) Inventors: Kunio Fukuda, Tokyo (JP); Isao Hidaka, Tokyo (JP)

(73) Assignee: Sony Corporation, Tokyo (JP)

(21) Appl. No.: 14/088,112

(22) Filed: Nov. 22, 2013

(30) Foreign Application Priority Data
Nov. 30, 2012 (JP) 2012-262699

(51) Int. Cl. A61B 5/021; A61B 5/0245

(52) U.S. Cl. CPC A61B 5/02125; A61B 5/0245
USPC 600/485

(57) ABSTRACT

There is provided a biological information measurement apparatus including a biological information acquisition unit configured to acquire at least first waveform information relating to a first waveform representing a beat of a measurement subject measured at a first measurement site, and as second waveform information relating to a second waveform representing the beat of the measurement subject measured at a second measurement site different from the first measurement site, information relating to a timing corresponding to a second feature which is a characteristic feature of the second waveform, and a pulse wave transit time calculation unit configured to, based on the first and second waveform information, calculate a pulse wave transit time, which is a difference between a timing corresponding to a first feature which is a characteristic feature of the first waveform and the timing corresponding to the second feature which is a characteristic feature of the second waveform.

### CROSS REFERENCE TO RELATED APPLICATIONS

This application claims the benefit of Japanese Priority Patent Application JP 2012-262699 filed Nov. 30, 2012, the entire contents of which are incorporated herein by reference.

### BACKGROUND

The present disclosure relates to a biological information measurement apparatus, a biological information measurement system, a biological information measurement method, and a program.

In the related art, as a method for measuring blood pressure, a direct measurement method has been known in which blood pressure is directly measured by utilizing air pressure. In this direct measurement method, pressure is applied on the blood vessels by supplying air through an air pump into a tube, called a cuff, that is wrapped around an arm or the like. By adjusting the air flow supplied into the cuff to change the pressure applied on the blood vessels, the pressure value when the blood starts or stop flowing is determined, whereby blood pressure is measured. However, since a direct measurement system blood pressure monitor has to have a cuff, an air pump, a detection device for detecting the start and stop of blood flowing and the like, such a monitor is not suited to portable applications. Further, since a direct measurement system blood pressure monitor takes effort and time for measurement, measuring blood pressure casually on a daily basis is difficult.

Accordingly, a so-called pulse wave system blood pressure monitor has been proposed that utilizes pulse wave velocity to measure blood pressure. For example, U.S. Patent Application Publication No. 2007/0276261 discloses a biological information monitoring apparatus that measures blood pressure by calculating pulse wave velocity based on an electrocardiography waveform (electrocardiogram) measured at the chest of a measurement subject (the user), and a pulse wave measured at a finger.

### SUMMARY

On the other hand, regarding blood pressure measurement, there is a large demand for constant measurement regardless of the time of day. For example, in order to grasp the symptoms exhibited by various diseases, in addition to the blood pressure values of the measurement subject during the day, monitoring the changes in blood pressure when asleep at night is thought to be important. In view of such circumstances, there is a demand for a blood pressure monitor capable of being operated at a lower power consumption.

According to an embodiment of the present disclosure, there are provided a novel and improved biological information measurement apparatus, a biological information measurement system, a biological information measurement method, and a program capable of measuring blood pressure at a lower power consumption.

According to an embodiment of the present disclosure, there is provided a biological information measurement apparatus including a biological information acquisition unit configured to acquire at least first waveform information relating to a first waveform representing a beat of a measurement subject measured at a first measurement site, and as second waveform information relating to a second waveform representing the beat of the measurement subject measured at a second measurement site different from the first measurement site, information relating to a timing corresponding to a second feature which is a characteristic feature of the second waveform, and a pulse wave transit time calculation unit configured to, based on the first waveform information and the second waveform information, calculate a pulse wave transit time, which is a difference between a timing corresponding to a first feature which is a characteristic feature of the first waveform and the timing corresponding to the second feature which is a characteristic feature of the second waveform.

According to an embodiment of the present disclosure, there is provided a biological information measurement system including a first waveform information measurement apparatus that includes a first waveform measurement unit configured to measure a first waveform representing a beat of a measurement subject at a first measurement site, and a first feature detection unit configured to detect a first feature which is a characteristic feature of the first waveform, and a second waveform information measurement apparatus that includes a second waveform measurement unit configured to measure a second waveform representing the beat of the measurement subject at a second measurement site that is different from the first measurement site, a second feature detection unit configured to detect a second feature which is a characteristic feature of the second waveform, and a biological information transmission unit configured to transmit second waveform information relating to the measured second waveform. The first waveform information measurement apparatus further includes a biological information reception unit configured to receive the second waveform information, and a pulse wave transit time calculation unit configured to calculate a pulse wave transit time, which is a difference between a timing corresponding to the first feature and the timing corresponding to the second feature. The biological information transmission unit is configured to transmit information relating to a timing corresponding to the second feature as the second waveform information.

According to an embodiment of the present disclosure, there is provided a biological information measurement method including acquiring at least first waveform information relating to a first waveform representing a beat of a measurement subject measured at a first measurement site, and as second waveform information relating to a second waveform representing the beat of the measurement subject measured at a second measurement site different from the first measurement site, information relating to a timing corresponding to a second feature which is a characteristic feature of the second waveform, and calculating, based on the first waveform information and the second waveform information, a pulse wave transit time, which is a difference between a timing corresponding to a first feature which is a characteristic feature of the first waveform and the timing corresponding to the second feature which is a characteristic feature of the second waveform.

According to an embodiment of the present disclosure, there is provided a program for causing a computer realize a function for acquiring at least first waveform information relating to a first waveform representing a beat of a measurement subject measured at a first measurement site, and as second waveform information relating to a second waveform representing the beat of the measurement subject measured at a second measurement site different from the first measurement site, information relating to a timing corresponding to a second feature which is a characteristic feature of the second waveform; and a function for calculating, based on the first waveform information and the second waveform information, a pulse wave transit time, which is a difference between a timing corresponding to a first feature which is a characteristic feature of the first waveform and the timing corresponding to the second feature which is a characteristic feature of the second waveform.

According to one or more embodiments of the present disclosure, at least first waveform information relating to a first waveform representing a beat of a measurement subject measured at a first measurement site, and as second waveform information relating to a second waveform representing the beat of the measurement subject measured at a second measurement site different from the first measurement site, information relating to a timing corresponding to a second feature which is a characteristic feature of the second waveform are acquired by a biological information acquisition unit. Further, based on the first waveform information and the second waveform information, a pulse wave transit time, which is a difference between a timing corresponding to a first feature, which is a characteristic feature of the first waveform, and the timing corresponding to the second feature, which is a characteristic feature of the second waveform, is calculated by a pulse wave transit time calculation unit. Therefore, of the information relating to the second waveform, since information relating to a timing corresponding to the second feature, which is a characteristic feature of the second waveform, is used, the amount of information that is handled during the series of processes is reduced.

Thus, according to one or more embodiments of the present disclosure, blood pressure can be measured at a lower power consumption.

### BRIEF DESCRIPTION OF THE DRAWINGS

FIG. 1 is an explanatory diagram illustrating pulse wave transit time;

FIG. 2 is an explanatory diagram illustrating a relationship between pulse wave velocity and blood pressure;

FIG. 3 is a schematic diagram illustrating an example of a usage method of a biological information measurement apparatus according to a first, a second, and a third embodiment of the present disclosure;

FIG. 4A is a schematic diagram illustrating an appearance example of an electrocardiogram information measurement apparatus according to a first, a second, and a third embodiment of the present disclosure;

FIG. 4B is a schematic diagram illustrating an appearance example of an electrocardiogram information measurement apparatus according to a first, a second, and a third embodiment of the present disclosure;

FIG. 5A is a schematic diagram illustrating an appearance example of a pulse wave information measurement apparatus according to a first, a second, and a third embodiment of the present disclosure;

FIG. 5B is a schematic diagram illustrating an appearance example of a pulse wave information measurement apparatus according to a first, a second, and a third embodiment of the present disclosure;

FIG. 6 is an explanatory diagram illustrating a pulse wave transit time calculation method according to a first embodiment of the present disclosure;

FIG. 7 is a schematic diagram illustrating a configuration example of a pulse wave detection packet;

FIG. 8 is a function block diagram illustrating a configuration example of an electrocardiogram information measurement apparatus according to a first embodiment of the present disclosure;

FIG. 9 is a function block diagram illustrating a configuration example of a pulse wave information measurement apparatus according to a first embodiment of the present disclosure;

FIG. 10 is a sequence diagram illustrating a pulse wave transit time calculation method according to a first embodiment of the present disclosure;

FIG. 11 is an explanatory diagram illustrating a pulse wave transit time calculation method according to a second embodiment of the present disclosure;

FIG. 12 is a function block diagram illustrating a configuration example of an electrocardiogram information measurement apparatus according to a second embodiment of the present disclosure;

FIG. 13 is a function block diagram illustrating a configuration example of a pulse wave information measurement apparatus according to a second embodiment of the present disclosure;

FIG. 14 is a sequence diagram illustrating a pulse wave transit time calculation method according to a second embodiment of the present disclosure;

FIG. 15 is an explanatory diagram illustrating a pulse wave transit time calculation method according to a third embodiment of the present disclosure;

FIG. 16 is a function block diagram illustrating a configuration example of a biological information measurement apparatus according to a third embodiment of the present disclosure;

FIG. 17 is a flow diagram illustrating a pulse wave transit time calculation method according to a third embodiment of the present disclosure;

FIG. 18 is a schematic diagram illustrating a configuration example of a heart sound measurement unit when a first waveform is a heart sound;

FIG. 19A is a schematic diagram illustrating an appearance example of a pulse wave information measurement apparatus when a second measurement site is a measurement subject's ear;

FIG. 19B is a schematic diagram illustrating an appearance example of a pulse wave information measurement apparatus when a second measurement site is a measurement subject's ear;

FIG. 19C is a schematic diagram illustrating an appearance example of a pulse wave information measurement apparatus when a second measurement site is a measurement subject's ear;

FIG. 20 is a function block diagram illustrating a configuration of a pulse wave information measurement apparatus when the pulse wave information measurement apparatus has a function for calculating a pulse wave transit time; and

FIG. 21 is a function block diagram illustrating an example of a hardware configuration of a biological information measurement apparatus according to a first, a second, and a third embodiment of the present disclosure.

### DETAILED DESCRIPTION OF THE EMBODIMENT(S)

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted. The description will now be made in the following order.

1. Pulse wave transit time
2. Apparatus appearance example and usage method
3. First embodiment of the present disclosure
3.1. Pulse wave transit time calculation method
3.2. Apparatus configuration
3.2.1. Electrocardiogram information measurement apparatus
3.2.2. Pulse wave information measurement apparatus
3.3. Pulse wave transit time measurement sequence
4. Second embodiment of the present disclosure
4.1. Pulse wave transit time calculation method
4.2. Apparatus configuration
4.2.1. Electrocardiogram information measurement apparatus
4.2.2. Pulse wave information measurement apparatus
4.3. Pulse wave transit time measurement sequence
5. First embodiment of the present disclosure
5.1. Pulse wave transit time calculation method
5.2. Apparatus configuration
5.3. Pulse wave transit time measurement sequence
6. Modified examples
6.1. First waveform
6.2. Pulse wave measurement site
6.3. Pulse wave transit time calculation unit
7. Hardware configuration

### 8. Conclusion

### 1. PULSE WAVE TRANSIT TIME

In a first, a second, and a third embodiment of the present disclosure, first, a first waveform representing a measurement subject's beat is measured at a first measurement site of the measurement subject. Further, a second waveform representing the measurement subject's beat is measured at a second measurement site that is different from the first site. Then, the measurement subject's blood pressure is measured by calculating the pulse wave transit time and the pulse wave velocity based on the measured first and second waveforms. It is noted that the pulse wave velocity is a value obtained by dividing the distance between the first measurement site and the second measurement site by the pulse wave transit time.

Here, a specific method for measuring blood pressure based on the pulse wave transit time will be described. It is noted that in the first, second, and third embodiments of the present disclosure, as an example of the first and second waveforms, when calculating the pulse wave transit time, an electrocardiogram waveform measured at the measurement subject's chest is used as the first waveform and a pulse wave measured at a finger of the measurement subject's hand is used as the second waveform. In the following description, unless stated otherwise, “electrocardiogram waveform” means “first waveform”, and “pulse wave” means “second waveform”.

However, in the present disclosure, the first and second waveforms are not limited to these examples. The first and second waveforms may be some other waveform, as long as they are waveforms that represent a beat of the measurement subject and were measured at different measurement sites to each other. Further, the measurement sites of the first and second waveforms are not limited to the chest and a finger on the hand, and they can be some other site on the human body. For example, the first waveform may be the measurement subject's heart sound. Still further, for example, if the second waveform is a pulse wave, then this pulse wave may be measured at the measurement subject's ear. Such modified examples of the first and second waveforms will be described in detail below in <6. Modified examples>.

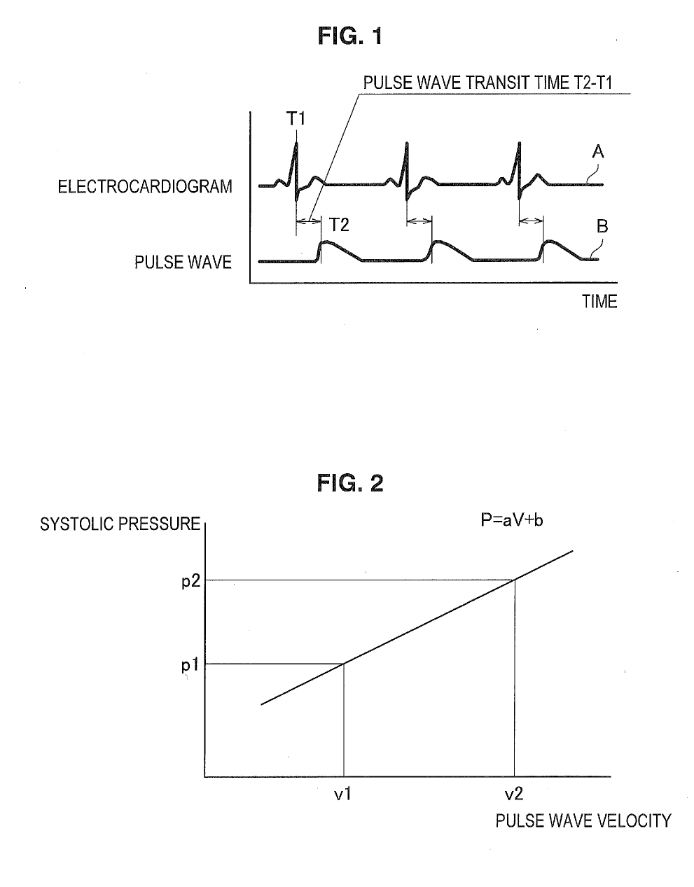

A method for measuring blood pressure based on the pulse wave transit time will now be described with reference to FIGS. 1 and 2. FIG. 1 is an explanatory diagram illustrating pulse wave transit time. Further, FIG. 2 is an explanatory diagram illustrating a relationship between pulse wave velocity and blood pressure.

As illustrated in FIG. 1, the change over time in the signal intensity of an electrocardiogram waveform A and the change over time in the signal intensity of a pulse wave B are plotted on a plane formed from a horizontal axis representing time and a vertical axis representing signal intensity. Here, as described above, pulse wave A is an example of the first waveform, and pulse wave B is an example of the second waveform.

If the time corresponding to a characteristic feature (first feature) of the periodic waveform of the electrocardiogram waveform A is T1, and the time corresponding to a characteristic feature (second feature) of the periodic waveform of the pulse wave B that appears after time T1 is T2, then the pulse wave transit time is defined as T2−T1. In the example illustrated in FIG. 1, the first feature is the initial rise point of the R wave in the electrocardiogram waveform A, and the second feature is the initial rise point of the pulse wave B. However, the first and second features are not limited to these examples, and they may be different characteristics of the electrocardiogram waveform A and the pulse wave B.

Further, the relationship between time T1 and time T2 does not have to be a relationship in which the blood sent from the heart at time T1 actually reaches the second measurement site where the pulse wave B is measured at time T2. As described below, since the correlation between the pulse wave transit time (velocity) and the blood pressure value can be obtained from the actual measured values of these two parameters, as long as the calculation method of pulse wave transit time (velocity) is fixed, there are no problems when calculating the blood pressure value.

Further, in FIG. 1, to facilitate the description, the graph is depicted with the signal intensity of the electrocardiogram waveform A having a greater value than the signal intensity of the pulse wave B. However, the relationship between the magnitude of the signal intensity of the electrocardiogram waveform and the magnitude of the signal intensity of the pulse wave B is not limited to this example. Namely, as long as the positional relationship on the horizontal axis (time) between time T1 and time T2 is clear, the vertical axis (signal intensity) scale is not especially limited. The signal intensity of the electrocardiogram waveform A and the signal intensity of the pulse wave B, for example, do not have to be plotted on the same vertical axis. In addition, the signal intensity of the electrocardiogram waveform A and the signal intensity of the pulse wave B can be subjected to processing with an appropriate filter, amplifier or the like after measurement so that the pulse wave transit time is calculated accurately.

Next, the relationship between pulse wave velocity and the systolic pressure (maximum blood pressure) value will be described with reference to FIG. 2. FIG. 2 is an explanatory diagram illustrating a relationship between pulse wave velocity and a systolic pressure (maximum blood pressure) value.

It is noted that, as described above, the pulse wave velocity is a value obtained by dividing the distance between the first measurement site and the second measurement site by the pulse wave transit time. In the examples illustrated in FIGS. 1 and 2, the pulse wave velocity is defined as the value obtained by dividing the distance from the measurement subject's chest where the electrocardiogram waveform A was measured and the finger on the measurement subject's hand where the pulse wave B was measured by the pulse wave transit time. It is noted that when the first measurement site is the chest and the second measurement site a finger on the measurement subject's hand, the distance between these can be presumed as being about ⅔ the height of the measurement subject.

As illustrated in FIG. 2, there is a linear relationship P=aV+b (wherein P represents systolic pressure value, V represents pulse wave velocity, and a and b represent constants) between pulse wave velocity and the systolic pressure value. Therefore, if the values of constants a and b are known, the systolic pressure value can be determined based on the pulse wave velocity calculated from the measured electrocardiogram waveform and pulse wave. However, since there are individual differences in the above-described linear relationship, the values of constants a and b are determined according to the measurement subject.

To determine the values of constants a and b, it is only necessary to know two arbitrary points on the straight line P=aV+b. Therefore, for example, using a direct measurement method, the measurement subject measures a pulse wave velocity v1 and a systolic pressure p1 with respect to v1 when the measurement subject is in a given state (first state). Next, the measurement subject measures a pulse wave velocity v2 and a systolic pressure p2 with respect to v2 when the measurement subject is in a different state (second state). Constants a and b can then be determined using the values for the pulse wave velocity v1 and v2 and the systolic pressure p1 and p2. In the following description, the determination in this manner of the values of constants a and b, namely, the linear relationship between pulse wave velocity and the systolic pressure (maximum blood pressure) value, will be referred to as calibration. Here, the first state and the second state are not especially limited, as long as they are states that produce a certain level of difference or more in the systolic pressure value of the measurement subject. For example, the first state may be a state before exercise (at rest), and the second state may be a state immediately after exercise.

Further, in the above description, although a case was described in which the pulse wave velocity v1 and v2 and the systolic pressure p1 and p2 were measured at different states, if it can be assumed that there are no large individual differences for constant a, which represents the gradient of the straight line, calibration can also be carried out with only data from a single point for the pulse wave velocity v1 and the systolic pressure p1. When carrying out calibration based on only one data point, an approximation formula based on the age of the measurement subject may be used, for example.

In addition, in the above description, although an example was described in which the pulse wave velocity v1 and v2 and the systolic pressure p1 and p2 were measured in two different states when performing calibration, there may be three or more measurement points. Namely, calibration can be performed based on pulse wave velocity v1, v2, v3 . . . and systolic pressure p1, p2, p3 . . . in three or more different states. The greater the number of measurement states, the more accurate calibration is.

In the above, a method was described with reference to FIGS. 1 and 2 for measuring blood pressure by utilizing an electrocardiogram waveform and a pulse wave of the measurement subject. It is noted that, among the series of processes for measuring the blood pressure, the following description will mainly be about a process for measuring an electrocardiogram waveform and a pulse wave of the measurement subject, and calculating the pulse wave transit time based on those measurement values. In the following description, since a known method can be used for the calibration processing to acquire the relationship between a pulse wave velocity and a systolic pressure value like that illustrated in FIG. 2, and for the processing to calculate the blood pressure value based on the pulse wave velocity using that relationship, a detailed description of such processing will be omitted here. It is also noted that the series of processing steps (pulse wave transit time calculation method) for measuring the electrocardiogram waveform and the pulse wave of the measurement subject and for calculating the pulse wave velocity based on those measured values will also be referred to as a biological information measurement method.

### 2. APPARATUS APPEARANCE EXAMPLE AND USAGE METHOD

Next, an appearance example of a biological information measurement apparatus according to the first, second, and third embodiments of the present disclosure will be described. Further, an example of a usage method of this biological information measurement apparatus will also be described.

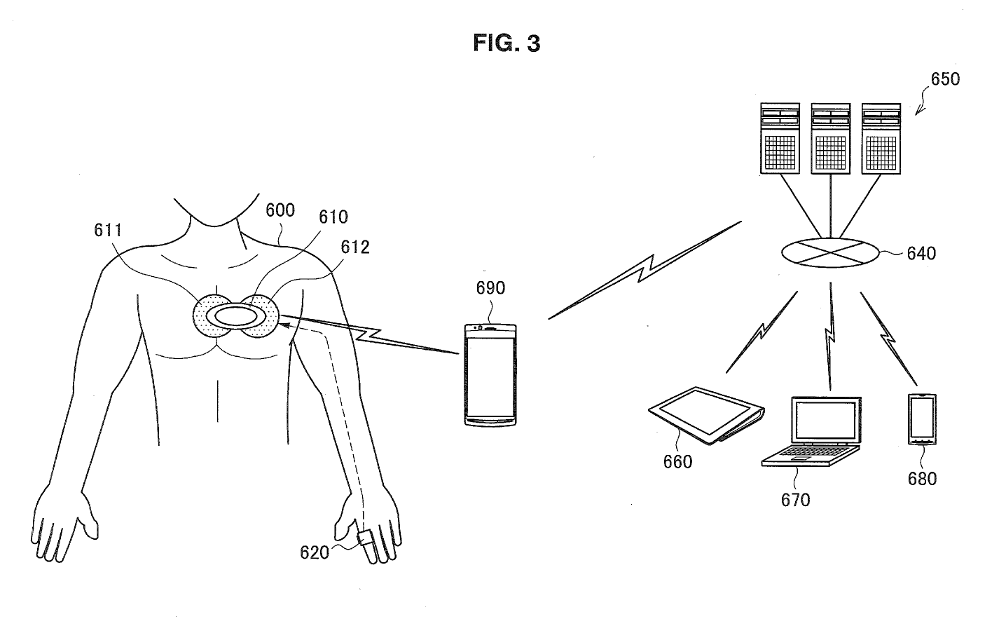

FIG. 3 is a schematic diagram illustrating an example of a usage method of the biological information measurement apparatus according to the first, second, and third embodiments of the present disclosure. As illustrated in FIG. 3, an electrocardiogram information measurement apparatus 610 is attached to the chest of a body 600 of the measurement subject, and a ring-type pulse wave information measurement apparatus 620 is fitted around a finger on the hand of the body 600.

Here, as described above, in the first, second, and third embodiments of the present disclosure, the measurement subject's blood pressure is measured based on an electrocardiogram waveform measured at the measurement subject's chest and a pulse wave measured at a finger on the measurement subject's hand. Although the first, second, and third embodiments of the present disclosure include a unit for measuring the electrocardiogram waveform (hereinafter referred to as “electrocardiogram sensor”) and a unit for measuring the pulse wave (hereinafter referred to as “pulse wave sensor”), in the present disclosure, these sensors can be some other apparatus, or these sensors may be integrally configured and incorporated in a single apparatus. It is noted that in the following description, if the electrocardiogram sensor and the pulse wave sensor are separate apparatuses, these sensors may also be referred to as an electrocardiogram information measurement apparatus and a pulse wave information measurement apparatus, respectively. Further, in the following description, if the electrocardiogram sensor and the pulse wave sensor are integrally configured and incorporated in a single apparatus, these sensors may also be referred to as an electrocardiogram measurement unit and a pulse wave measurement unit, respectively. Namely, in the following description, the term electrocardiogram sensor refers to at least either an electrocardiogram information measurement apparatus or an electrocardiogram measurement unit, and the term pulse wave sensor refers to at least either a pulse wave information measurement apparatus or a pulse wave measurement unit.

In the example illustrated in FIG. 3, a case is illustrated in which the electrocardiogram sensor (electrocardiogram information measurement apparatus 610) and the pulse wave sensor (pulse wave information measurement apparatus 620) are separate apparatuses. Thus, if the electrocardiogram sensor and the pulse wave sensor are separate apparatuses, in the following description, in some cases at least either the electrocardiogram information measurement apparatus or the pulse wave information measurement apparatus may be referred to as a biological information measurement apparatus. On the other hand, if the electrocardiogram sensor and the pulse wave sensor are integrally configured and incorporated in a single apparatus, in the following description, that apparatus may be referred to as a biological information measurement apparatus.

In addition, if the electrocardiogram sensor and the pulse wave sensor are separate apparatuses, information relating to the measurement result may be transmitted to either of the sensors, and the pulse wave transit time calculated by that sensor. It is noted that the function for calculating the pulse wave transit time may be included in just the electrocardiogram sensor or just the pulse wave sensor, or may be included in both of these sensors.

Here, a case in which the electrocardiogram information measurement apparatus 610 has the function for calculating the pulse wave transit time will be described with reference to FIG. 3 as an example of a usage method of the biological information measurement apparatus according to the first, second, and third embodiments of the present disclosure. Namely, in the example illustrated in FIG. 3, a case is illustrated in which information relating to the pulse wave of the measurement subject measured by the pulse wave information measurement apparatus 620 is transmitted to the electrocardiogram information measurement apparatus 610, and the electrocardiogram information measurement apparatus 610 calculates the pulse wave transit time based on that pulse wave information and electrocardiogram information relating to the electrocardiogram waveform that it itself measured.

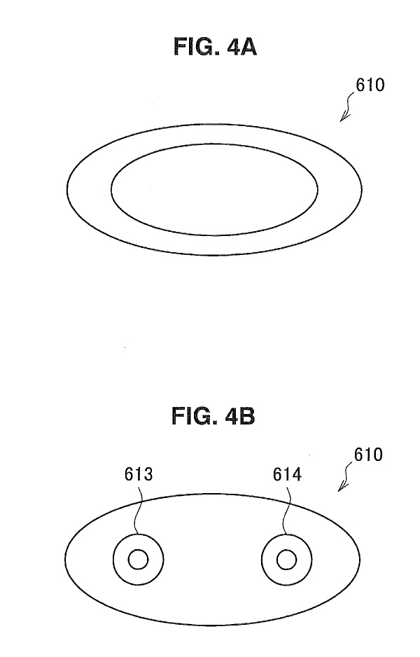

As illustrated in FIG. 3, the electrocardiogram information measurement apparatus 610 is attached to the chest of the measurement subject. Specifically, a pair of moist electrocardiogram measurement electrodes (electrocardiogram electrodes) 611 and 612 that have a suction force are provided on a face of the electrocardiogram information measurement apparatus 610. The electrocardiogram information measurement apparatus 610 is attached to the chest of the measurement subject's body 600 by these electrodes 611 and 612. The electrocardiogram information measurement apparatus 610 acquires electrocardiogram information relating to the electrocardiogram waveform by measuring the electrocardiogram waveform of the measurement subject using the electrodes 611 and 612. Here, the electrocardiogram information may also include information relating to the timing corresponding to the first feature, which is a characteristic feature of the electrocardiogram waveform. The first feature may be, for example, the initial rise or initial fall of a P wave, a Q wave, an R wave, an S wave, or a T wave included in the electrocardiogram waveform. It is noted that the configuration of the electrocardiogram information measurement apparatus 610 will be described below with reference to FIGS. 4A and 4B.

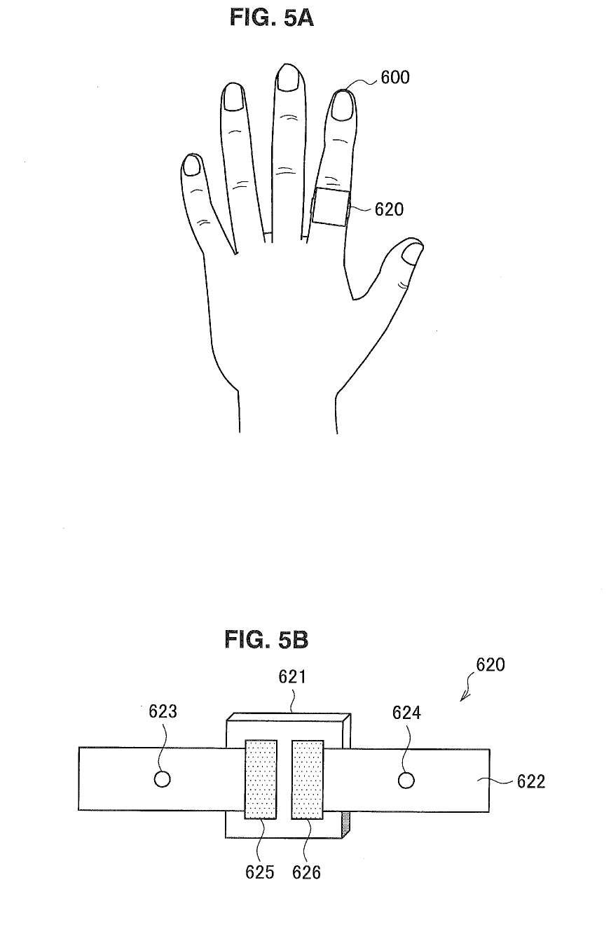

The pulse wave information measurement apparatus 620 is fitted to a finger on the measurement subject's hand. The pulse wave information measurement apparatus 620 acquires pulse wave information relating to the pulse wave by measuring the pulse wave of the measurement subject. Here, the pulse wave information may also include information relating to the timing corresponding to the second feature, which is a characteristic feature of the pulse wave. The second feature may be, for example, the initial rise of the pulse wave. It is noted that the configuration of the pulse wave information measurement apparatus 620 will be described below with reference to FIGS. 5A and 5B.

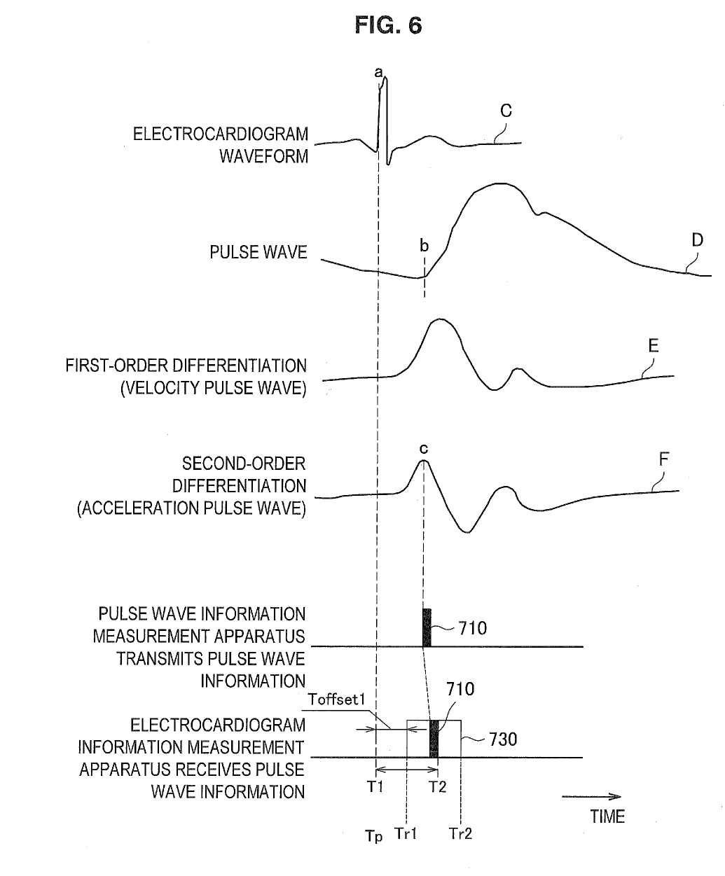

The pulse wave information measurement apparatus 620 transmits the measured pulse wave information relating to the pulse wave to the electrocardiogram information measurement apparatus 610. Here, the pulse wave information measurement apparatus 620 does not transmit the pulse wave itself as the pulse wave information, rather it transmits information relating to the timing corresponding to the second feature. Further, the pulse wave information measurement apparatus 620 may also transmit pulse wave information that also includes a predetermined time, for example, pulse wave information that includes the pulse rate for a one minute duration. It is noted that the details of the transmission of information between the pulse wave information measurement apparatus 620 and the electrocardiogram information measurement apparatus 610 will be described below for each embodiment of the present disclosure with reference to FIGS. 6, 11, and 15.

Here, in the example illustrated in FIG. 3, rather than using a cable or the like, human body communication (HBC) is used for the communication between the electrocardiogram information measurement apparatus 610 and the pulse wave information measurement apparatus 620. Examples of human body communication methods include an electric field method in which communication is performed by producing an electric field on the surface of the human body, and a current method in which communication is performed by applying a minute current. In the first and second embodiments of the present disclosure, human body communication based on the electric field method is used for communication between the electrocardiogram information measurement apparatus 610 and the pulse wave information measurement apparatus 620. Regarding electric field method type human body communication, IEEE 802.15.6, which is a BAN (body area network) standard may be employed as one standardized method.

It is noted that the present disclosure is not limited to this example. The communication between the electrocardiogram information measurement apparatus 610 and the pulse wave information measurement apparatus 620 may be carried out based on human body communication that employs the current method. Further, the communication between the electrocardiogram information measurement apparatus 610 and the pulse wave information measurement apparatus 620 can also be carried out by some other wired or wireless communication method. However, using human body communication enables communication to be performed at a lower power consumption than other wireless communication methods. The electrocardiogram information measurement apparatus 610 calculates the pulse wave transit time based on the electrocardiogram information it itself acquired and the received pulse wave information. Specifically, the electrocardiogram information measurement apparatus 610 calculates the pulse wave transit time by taking the difference between a timing T1 corresponding to the first feature included in the electrocardiogram information and a timing T2 corresponding to the second feature included in the pulse wave information.

In addition, the electrocardiogram information measurement apparatus 610 includes a communication unit for communicating with an external device. The electrocardiogram information measurement apparatus 610 transmits information relating to the pulse wave transit time to a mobile terminal 690, for example, via this communication unit. As the communication method for transmitting the information relating to the pulse wave transit time to the mobile terminal 690 from the electrocardiogram information measurement apparatus 610, for example, a known wireless communication method, such as Bluetooth®, is used. Further, the mobile terminal 690 may be an external device that can be carried around by the measurement subject, such as a smartphone, for example.

Calibration data about the measurement subject for obtaining a relationship between the pulse wave velocity and the systolic pressure value like that illustrated in FIG. 2, and physical data, such as the height and weight of the measurement subject, for example, are input in the mobile terminal 690 in advance. Based on this data, the mobile terminal 690 calculates the pulse wave velocity from the pulse wave transit time transmitted from the electrocardiogram information measurement apparatus 610. Further, from the calibration the mobile terminal 690 obtains the relationship between the pulse wave velocity and the systolic pressure value, and performs processing for calculating the blood pressure value of the measurement subject from the calculated pulse wave velocity. Calculating blood pressure with a device that is carried around by the measurement subject like the mobile terminal 690 enables the measurement subject to confirm his/her blood pressure at a desired timing, so that user friendliness for the measurement subject is improved.

It is noted that the calculation of the pulse wave transit time may be performed by the mobile terminal 690 rather than by the electrocardiogram information measurement apparatus 610. If calculating the pulse wave transit time with the mobile terminal 690, the electrocardiogram information measurement apparatus 610 may also transmit information relating to the timing T1 corresponding to the first feature and information relating to the timing T2 corresponding to the second feature. Further, the electrocardiogram information measurement apparatus 610 may also transmit as necessary various other types of information to the mobile terminal 690, such as the data of the electrocardiogram waveform per se.

The mobile terminal 690 stores biological information, such as information relating to the measured pulse wave (pulse) and electrocardiogram waveform, as well as information relating to the calculated blood pressure, in a storage medium included therein. Further, the mobile terminal 690 transmits this biological information to a server 650, for example, via a communication network 640. The server 650 can store the received biological information about the measurement subject for a predetermined period of time. Here, the biological information may be any information about the biological activity of the measurement subject. Although in the example illustrated in FIG. 3, information relating to the electrocardiogram waveform, information relating to the pulse wave, and the blood pressure value are handled as the biological information, the biological information can also include, for example, information relating to heart sound, breathing, body temperature and the like.

The biological information stored in the server 650 can be viewed using a plurality of devices different from each other, such as a tablet terminal 660, a laptop PC 670, and another mobile terminal 680, for example. Therefore, the biological information about the measurement subject can be shared by medical staff and caregivers, for example, enabling the measurement subject's state of health and medical condition to be managed.

In the above, an example of the usage method of the biological information measurement apparatus according to the first, second, and third embodiments of the present disclosure was described with reference to FIG. 3. It is noted that the server 650 illustrated in FIG. 3 is an example of storage (a storage apparatus) on a network. However, the apparatus in which the biological information about the measurement subject is stored is not limited to this example, and the biological information can be stored in any known storage apparatus. Further, the tablet terminal 660, the laptop PC 670, and the other mobile terminal 680 are examples of devices for viewing the biological information about the measurement subject. However, the device for viewing the biological information about the measurement subject is not limited to these examples, any other device can be used.

Next, an appearance example of the electrocardiogram information measurement apparatus 610 and of the pulse wave information measurement apparatus 620 will be described with reference to FIGS. 4A, 4B, 5A, and 5B. FIGS. 4A and 4B are schematic diagrams illustrating an appearance example of the electrocardiogram information measurement apparatus 610. FIGS. 5A and 5B are schematic diagrams illustrating an appearance example of the pulse wave information measurement apparatus 620. It is noted that the electrocardiogram information measurement apparatus 610 and the pulse wave information measurement apparatus 620 illustrated in FIGS. 4A, 4B, 5A, and 5B correspond to the case illustrated in FIG. 3 in which the electrocardiogram sensor and the pulse wave sensor are separate apparatuses.

As illustrated in FIGS. 4A and 4B, the electrocardiogram information measurement apparatus 610 has a disc-shaped appearance in which elliptical flat plates oppose each other. The electrocardiogram information measurement apparatus 610 is used by bringing one of its faces into contact with the human body as a patch-type measurement apparatus. FIG. 4A illustrates a front face, which is one of the faces of the disc, and FIG. 4B illustrates a rear face, which the face opposing the front face.

A pair of connectors 613 and 614 for the electrodes used in electrocardiogram measurement is provided on the rear face of the electrocardiogram information measurement apparatus 610. An electrode is mounted on the connectors 613 and 614. The measurement subject's electrocardiogram is measured as a potential difference between the electrodes by bringing the electrodes into contact with the measurement subject's body 600. The potential difference value measured as the electrocardiogram is about several mV.

As illustrated above, in the example illustrated in FIG. 3, a pair of moist electrodes 611 and 612 that have a suction force are mounted on the connectors 613 and 614. It is noted that the electrodes mounted on the connectors 613 and 614 are not limited to this example, various types of electrode may be used according to the application. However, to constantly perform electrocardiogram measurement, it is preferred to use electrodes that have a suction force like those illustrated in FIG. 3.

In the above, an appearance example of the electrocardiogram information measurement apparatus 610 was described with reference to FIGS. 4A and 4B. It is noted that although in the above-described description the appearance of the electrocardiogram information measurement apparatus 610 was a disc shape, the electrocardiogram information measurement apparatus 610 according to an embodiment of the present disclosure is not limited to this example. The appearance of the electrocardiogram information measurement apparatus 610 is not especially limited, and may be any shape, as long as the electrocardiogram information measurement apparatus 610 has the above-described function for measuring the electrocardiogram waveform and the function for communicating with the pulse wave information measurement apparatus 620. However, in consideration of the fact that the electrocardiogram information measurement apparatus 610 is constantly carried around (attached to) by the measurement subject, it is preferred that the appearance of the electrocardiogram information measurement apparatus 610 has a shape that improves user friendliness for the measurement subject.

As illustrated in FIGS. 5A and 5B, the pulse wave information measurement apparatus 620 has a ring-like appearance, and is worn on a finger on the measurement subject's hand. The pulse wave information measurement apparatus 620 has a roughly cuboid main body 621 and a belt 622 for fixing the main body 621 to the measurement subject's finger. The pulse wave information measurement apparatus 620 is fixed to the finger by winding the belt 622 around the measurement subject's finger so that one face of the main body 621 is in contact with the finger.

A light-emitting element 623 and a light-receiving element 624 are provided on the face of the belt 622 that is in contact with the measurement subject's finger. The light-emitting element 623 is, for example, a light-emitting diode (LED) that irradiates infrared light. Further, the light-receiving element 624 is, for example, a photodiode. When infrared light is irradiated from the light-emitting element 623 with the belt 622 wrapped around the measurement subject's finger, the infrared light passes through or is scattered by the measurement subject's finger, and reaches the light-receiving element 624. Namely, the light-emitting element 623 and the light-receiving element 624 are provided at positions that allow the light that has passed through or been reflected by the measurement subject's finger among the light irradiated from the light-emitting element 623 to be detected by the light-receiving element 624 when the belt 622 is wrapped around the measurement subject's finger.

Here, generally, the hemoglobin present in a person's blood tends to absorb light in a specific wavelength, for example, infrared light. Since the level of hemoglobin is proportional to the amount of blood flowing through an artery, when light having a specific wavelength is irradiated on a pulse wave detection site (the finger), and the light that has passed though or been reflected is detected, the amount of light that is detected also changes based on the blood flow at the pulse wave detection site. Therefore, changes in the blood flow in the blood vessel can be detected from this detected amount of light, so that the pulse wave can be measured.

Further, a pair of electrodes 626 and 627 is provided at a site of the main body 621 that is in contact with the measurement subject's finger. These electrodes 626 and 627 play the role of transmitting information when communicating with the human body.

In the above, an appearance example of the pulse wave information measurement apparatus 620 was described with reference to FIGS. 5A and 5B. It is noted that although in the above-described description the appearance of the pulse wave information measurement apparatus 620 was a ring-like shape, the pulse wave information measurement apparatus 620 according to an embodiment of the present disclosure is not limited to this example. For example, as long as the pulse wave information measurement apparatus 620 has the above-described function for measuring the pulse wave and the function for communicating with the electrocardiogram information measurement apparatus 610, the pulse wave information measurement apparatus 620 may be a type that sandwiches a site on the body, or a wristwatch type that is wrapped around the arm rather than the measurement subject's finger.

In the above, appearance examples of the electrocardiogram information measurement apparatus 610 and of the pulse wave information measurement apparatus 620 were described with reference to FIGS. 4A, 4B, 5A, and 5B.

It is noted that if the unit for measuring the electrocardiogram waveform and the unit for measuring the pulse wave integrally configured and incorporated in a single apparatus (a biological information measurement apparatus), for example, the electrocardiogram information measurement apparatus 610 and pulse wave information measurement apparatus 620 illustrated in FIG. 3 can also function as the electrocardiogram measurement unit and the pulse wave measurement unit of the biological information measurement apparatus.

Further, alternatively, if the unit for measuring the electrocardiogram waveform and the unit for measuring the pulse wave are integrally configured and incorporated in a single apparatus (a biological information measurement apparatus), the electrocardiogram information measurement apparatus 610 illustrated in FIGS. 4A and 4B may include the functions of the pulse wave information measurement apparatus 620. In such a case, the electrocardiogram information measurement apparatus 610 may further include a structure for measuring the pulse wave of the measurement subject. For example, in the appearance illustrated in FIGS. 4A and 4B, the electrocardiogram information measurement apparatus 610 may further have a pulse wave detection window for measuring the pulse wave, so that the pulse wave is detected by the measurement subject pressing a finger against the pulse wave detection window.

In the following, the first, second, and third embodiments of the present disclosure will be described in more detail.

### 3. FIRST EMBODIMENT OF THE PRESENT DISCLOSURE

Next, a first embodiment of the present disclosure will be described. In the first embodiment of the present disclosure, an electrocardiogram sensor (electrocardiogram information measurement apparatus) and a pulse wave sensor (pulse wave information measurement apparatus) are configured as separate apparatuses. Further, in the first embodiment of the present disclosure, the apparatus having the function for calculating the pulse wave transit time is the electrocardiogram information measurement apparatus. The electrocardiogram information measurement apparatus calculates the pulse wave transit time based on pulse wave information transmitted from the pulse wave information measurement apparatus. Namely, the electrocardiogram information measurement apparatus 610 and the pulse wave information measurement apparatus 620 described with reference to FIG. 3 correspond to the first embodiment of the present disclosure.

### (3.1. Pulse Wave Transit Time Calculation Method)

First, the method for calculating the pulse wave transit time according to the first embodiment of the present disclosure will be specifically described with reference to FIG. 6. FIG. 6 is an explanatory diagram illustrating a method for calculating the pulse wave transit time according to the first embodiment of the present disclosure. In FIG. 6, the timing at which pulse wave information is transmitted from the pulse wave information measurement apparatus to the electrocardiogram information measurement apparatus is illustrated in association with the electrocardiogram waveform and the pulse wave, with the horizontal axis representing time.

As illustrated in FIG. 6, an electrocardiogram waveform C, a pulse wave D, a velocity pulse wave E, and an acceleration pulse wave F are on the same time axis. Here, the velocity pulse wave E is a waveform obtained by differentiating the pulse wave D once with respect to time, and the acceleration pulse wave F is a waveform obtained by differentiating the pulse wave D twice with respect to time.

First, the electrocardiogram information measurement apparatus detects a first feature and the timing corresponding to that first feature from the electrocardiogram waveform C that it itself measured. In the example illustrated in FIG. 6, using an initial rise point a of the R wave in the electrocardiogram waveform C as the first feature, the timing T1 corresponding to the initial rise point a is detected. However, in the present embodiment, the first feature is not limited to this example, some other point in the electrocardiogram waveform C may be used. For example, the first feature may be an initial fall of the P wave, the Q wave, the S wave, or the T wave in the electrocardiogram waveform C.

Next, the pulse wave information measurement apparatus detects a second feature and the timing corresponding to that second feature from the pulse wave D that it itself measured. In the example illustrated in FIG. 6, an initial rise point b of the pulse wave D is detected as the second feature. Here, as illustrated in FIG. 6, in order to detect the second feature, the pulse wave information measurement apparatus can also differentiate the pulse wave D with respect to time. Since the initial rise point b of the pulse wave D matches a point c that gives the local maximum value for the acceleration pulse wave F obtained by differentiating the pulse wave D twice with respect to time, the pulse wave information measurement apparatus can detect the initial rise point b of the pulse wave by determining the point c that gives the local maximum value for the acceleration pulse wave F. Since it can be difficult to detect the initial rise point b of the pulse wave D from the pulse wave D, by thus utilizing the acceleration pulse wave F, the pulse wave information measurement apparatus can more accurately detect the initial rise point b of the pulse wave D.

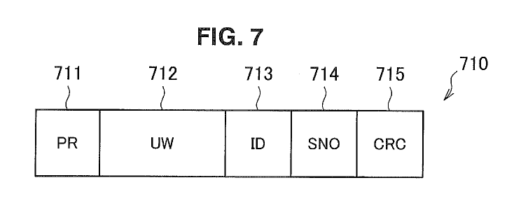

When the pulse wave information measurement apparatus detects the initial rise point b of the pulse wave D, it transmits pulse wave information, which is information relating to the pulse wave, to the electrocardiogram information measurement apparatus via a biological information transmission unit. It is noted that human body communication, for example, is used for this transmission and reception of the pulse wave information. Here, in the present embodiment, information relating to the timing corresponding to the initial rise point b of the pulse wave D is used as the pulse wave information, rather than all the information relating to the pulse wave D (the waveform data itself). In the example illustrated in FIG. 6, the pulse wave information measurement apparatus transmits to the electrocardiogram information measurement apparatus a pulse wave detection packet 710 as the pulse wave information. The pulse wave detection packet 710 is data in packet units indicating that the initial rise point b has been detected by the pulse wave information measurement apparatus from the pulse wave D. Namely, the pulse wave detection packet 710 can be said to be information relating to the timing corresponding to the initial rise point b of the pulse wave D. Thus, by transmitting and receiving only information relating to the timing corresponding to the initial rise point b of the pulse wave D rather than all the information relating to the pulse wave D, the amount of data that is handled can be reduced, and a decrease in power consumption of the electrocardiogram information measurement apparatus and the pulse wave information measurement apparatus can be realized. It is noted that the configuration of the pulse wave detection packet 710 will be described below in more detail with reference to FIG. 7.

Further, the pulse wave information measurement apparatus starts up the biological information transmission unit for just the period that the pulse wave detection packet 710 is being transmitted. At other times the biological information transmission unit may be in a sleep state. Namely, the pulse wave information measurement apparatus can start up the biological information transmission unit for just a limited time.

The electrocardiogram information measurement apparatus calculates a pulse wave transit time Tp from the relationship Tp=T2−T1 by utilizing a time T2 at which the pulse wave detection packet 710 transmitted from the pulse wave information measurement apparatus is received. It is noted that, as described above, the transmission of the pulse wave detection packet 710 from the pulse wave information measurement apparatus to the electrocardiogram information measurement apparatus is performed utilizing human body communication, for example. Further, as described below with reference to FIG. 7, the pulse wave detection packet 710 includes an error detection code (CRC) 715. Based on the error detection code (CRC) 715, the electrocardiogram information measurement apparatus can determine whether reception of the pulse wave detection packet 710 was performed normally or not. If an error was detected based on the error detection code (CRC) 715, the electrocardiogram information measurement apparatus does not calculate the pulse wave transit time, and waits for the pulse wave detection packet 710 based on the initial rise point b detected from the next beat of the pulse wave D.

Here, the electrocardiogram information measurement apparatus may include a biological information reception unit for receiving the pulse wave detection packet 710. This biological information reception unit can be can be configured so that it is started up ony during a pulse wave information reception period, which is a predetermined duration, and receives the pulse wave detection packet 710 during this pulse wave information reception period. In the example illustrated in FIG. 6, a case is illustrated in which, in the electrocardiogram information measurement apparatus, the biological information reception unit is started up during a period from time Tr1 to Tr2, and the pulse wave detection packet 710 is received during this period. In the following description, this pulse wave information reception period, which is the time that the biological information reception unit is started up, will be referred to as a reception window 730. By providing the reception window 730 and limiting the running time of the biological information reception unit, the power consumption of the electrocardiogram information measurement apparatus can be reduced even further. It is noted that if the pulse wave detection packet 710 is not received during the reception window 730, the electrocardiogram information measurement apparatus does not calculate the pulse wave transit time, and waits for the pulse wave detection packet 710 based on the initial rise point b detected from the next beat of the pulse wave D.

The time Tr1 that acts as a base point for the reception window 730 and the width (Tr2−Tr1) of the reception window 730 are determined based on the timing T1 corresponding to the initial rise point a of the R wave in the electrocardiogram wavelength C, for example. Specifically, the value of the pulse wave transit time of the measurement subject is estimated based on a previous pulse wave transit time measurement value or based on a statistic obtained from past pulse wave transit time measurement values, for example. Based on this predicted value, the center value of the reception window 730 and the width of the reception window 730 may be determined Pulse wave transit time is known to usually be about 200 ms, for example. Further, when the measurement subject's pulse wave transit time is continuously measured, it is known that the pulse wave transit time does not greatly change. Therefore, as a specific example of the reception window 730, using T1 as a base point, Tr1 may be set at a point 100 ms from T1, and Tr2 may be set at a point 300 ms after T1.

In the above example, the fact that the width of the reception window 730 is 200 nm and the fact that an interval Toffset between Tr1 and T1 is 100 ms are input in advance in the electrocardiogram information measurement apparatus. Further, when the electrocardiogram information measurement apparatus detects the timing T1 corresponding to the initial rise point a of the R wave from the electrocardiogram waveform C it itself measured, the electrocardiogram information measurement apparatus starts up the biological information reception unit 100 ms after T1 (after Tr1), and switches the biological information reception unit to a sleep state 300 ms after T1 (after Tr2). By operating in this manner, the electrocardiogram information measurement apparatus can start up the biological information transmission unit for just a limited time. It is noted that the values for Tr1 and Tr2 in the above example are merely examples. The values of Tr1 and Tr2 may be appropriately set based on a measurement subject previous pulse wave transit time measurement value of the measurement subject or based on a statistic obtained from past pulse wave transit time measurement values of the measurement subject, for example.

It is noted that, as illustrated in FIG. 6, a time lag caused by the time taken to transmit the pulse wave detection packet 710 and the time taken by the biological information reception unit to receive the pulse wave detection packet 710 is produced between the timing corresponding to the actual initial rise point b of the pulse wave D and the timing T2 when the pulse wave detection packet 710 is received. However, since the transmission speed of human body communication transmitting the pulse wave detection packet 710 is about 100 kbps, and since the packet length of the pulse wave detection packet 710 is, as described below with reference to FIG. 7, about 50 bits, this time lag is about 500 ns. As described above, since pulse wave transit time is usually about 200 ms, if the time lag is at this level, the effect on the ultimately-calculated blood pressure value can be ignored. It is noted that if the time lag can be predicted in advance, processing can be carried out to correct that time lag amount when calculating the pulse wave transit time.

Here, the configuration of the pulse wave detection packet 710 will be described with reference to FIG. 7. FIG. 7 is a schematic diagram illustrating a configuration example of the pulse wave detection packet 710.

The pulse wave detection packet 710 has a data amount of about 50 bits, for example. As illustrated in FIG. 7, the pulse wave detection packet 710 is configured from a preamble (PR) 711 for performing bit synchronization, a unique word (UW) 712 indicating the start of the data, a pulse wave information measurement apparatus ID number (ID) 713, a pulse wave sequence number (SO) 714, and an error detection code (CRC) 715.

In the first embodiment of the present disclosure, communication between the electrocardiogram information measurement apparatus and the pulse wave information measurement apparatus is only carried out in one direction from the pulse wave information measurement apparatus to the electrocardiogram information measurement apparatus by human body communication, for example. Moreover, re-transmission of the pulse wave detection packet 710 is not carried out. Therefore, if an error is detected based on the error detection code (CRC) 715, the electrocardiogram information measurement apparatus discards that pulse wave detection packet 710, and does not calculate the pulse wave transit time. Also, the electrocardiogram information measurement apparatus does not calculate the pulse wave transit time if the pulse wave detection packet 710 is not received during the reception window 730.

The sequence number 714 has a 16-bit data amount, for example, which changes in the range of 0 to 15. Specifically, the value of the sequence number 714 increases by one each time a pulse wave detection packet 710 is transmitted. When this value reaches 15, the value starts again from zero. By changing the sequence number 714 in this manner, the electrocardiogram information measurement apparatus can find gaps in the data by referring to the sequence number 714 included in the received pulse wave detection packet 710.

In the above, the method for calculating the pulse wave transit time according to the first embodiment of the present disclosure was described with reference to FIGS. 6 and 7. Next, the specific configuration for realizing this method will be described.

### (3.2. Apparatus Configuration)

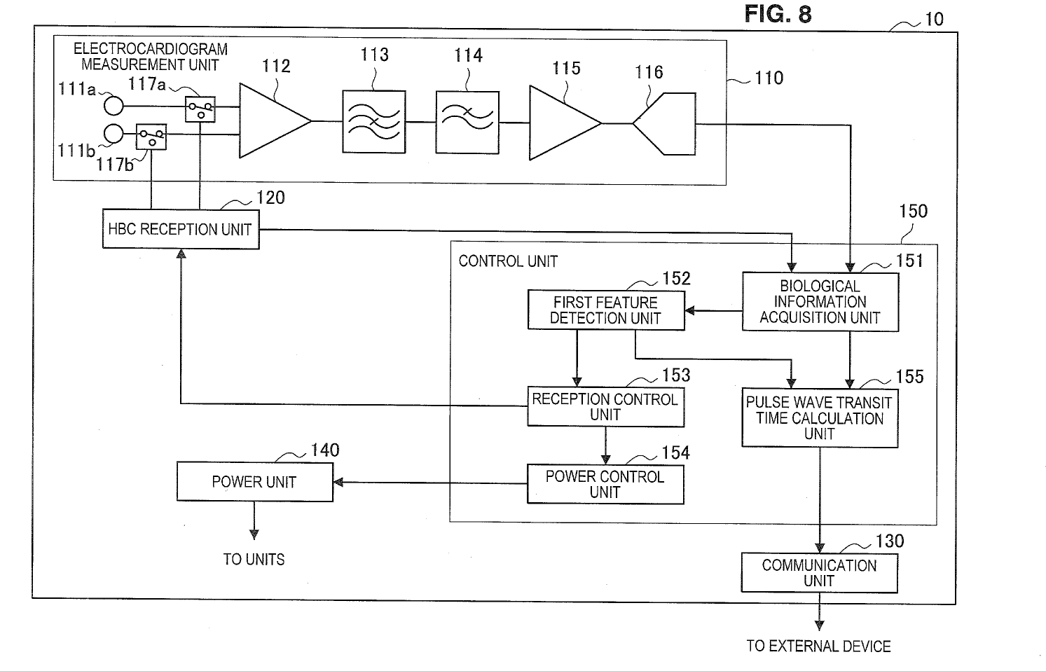

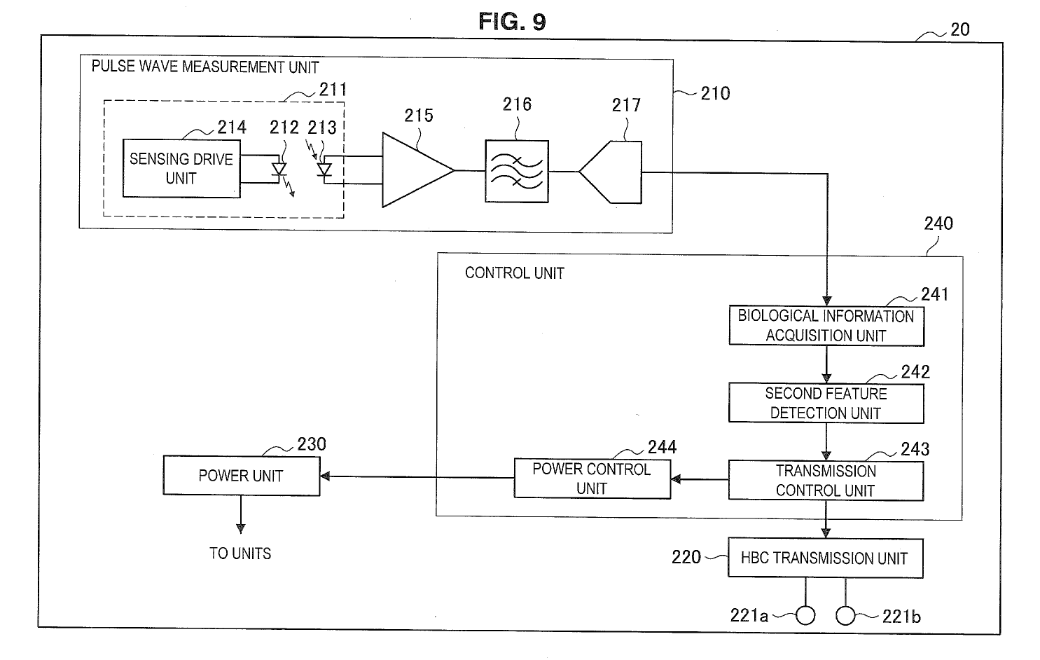

The configuration of the electrocardiogram information measurement apparatus and the pulse wave information measurement apparatus according to the first embodiment of the present disclosure will be specifically described with reference to FIGS. 8 and 9. FIG. 8 is a function block diagram illustrating a configuration example of the electrocardiogram information measurement apparatus according to the first embodiment of the present disclosure. Further, FIG. 9 is a function block diagram illustrating a configuration example of the pulse wave information measurement apparatus according to the first embodiment of the present disclosure.

### (3.2.1. Electrocardiogram Information Measurement Apparatus)

As illustrated in FIG. 8, an electrocardiogram information measurement apparatus 10 according to the first embodiment of the present disclosure includes an electrocardiogram measurement unit 110, an HBC reception unit 120, a communication unit 130, a power unit 140, and a control unit 150. The electrocardiogram measurement unit 110 measures an electrocardiogram of the measurement subject at a first measurement site of the measurement subject. Specifically, the electrocardiogram measurement unit 110, which has a pair of below-described electrodes 111a and 111b, measures the electrocardiogram waveform of the measurement subject as a potential difference between the electrodes 111a and 111b when the electrodes 111a and 111b are brought into contact with the measurement subject's chest. The electrocardiogram measurement unit 110 transmits the data regarding the measured electrocardiogram waveform to a below-described biological information acquisition unit 151 in the control unit 150.

Here, the specific configuration of the electrocardiogram measurement unit 110 will be described. The electrocardiogram measurement unit 110 includes electrodes 111a and 111b, a differential amplifier 112, a notch filter 113, a low-pass filter 114, an amplifier 115, an analog-digital converter (AD converter) 116, and switches 117a and 117b.

The electrodes 111a and 111b are brought into contact with the measurement subject's chest, and the potential difference between the two electrodes is measured. Electrocardiogram measurement is performed by measuring the potential difference between a desired two points on the human body. Therefore, by measuring the change with respect to time in the potential difference between the electrodes 111a and 111b, the electrocardiogram waveform of the measurement subject can be measured. The electrodes 111a and 111b correspond to the electrodes 611 and 612 illustrated in FIG. 3, for example.

The differential amplifier 112 amplifies the potential difference measured between the electrodes 111a and 111b. Generally, the potential difference measured between the electrodes 111a and 111b is about a few mV. The differential amplifier 112 is designed to amplify this potential difference by about 100 times, for example.

The notch filter 113 and the low-pass filter 114 are filters for removing unwanted noise from the signal amplified by the differential amplifier 112. The notch filter 113 is a filter circuit for reducing the frequency component in a specific band. In the present embodiment, in view of the effects from a commercial alternating power supply that is located near the electrocardiogram measurement unit 110, the notch filter 113 is designed to reduce the frequency component near the 50 Hz or the 60 Hz band. Further, the low-pass filter 114 is a filter circuit for reducing noise over a wide band that is not used in electrocardiogram measurement. In the present embodiment, the low-pass filter 114 is set so that its cutoff frequency is about 100 Hz, in view of the fact that the frequency that the electrocardiogram waveform has is about a few Hz.

Here, the removal of unwanted signals can also be appropriately carried out in a subsequent signal processing process (signal processing process by the 150). Therefore, the characteristics of the notch filter 113 and the low-pass filter 114 can be freely designed as long as their level of noise reduction is appropriate for an amplification system.

The amplifier 115 amplifies the signal from which unwanted noise has been reduced by the notch filter 113 and the low-pass filter 114. The gain by the amplifier 115 is set at about 10-fold, for example. Therefore, for example, a potential difference between the electrodes 111a and 111b that was about a few mV is amplified to about a few hundred mV to 1 V, and ultimately input to the AD converter 116.

The AD converter 116 converts (AD converts) the input signal, namely, a signal relating to the amplified electrocardiogram waveform, from an analog signal into a digital signal, and transmits the converted signal to the biological information acquisition unit 151 in the control unit 150 as a digital signal.

The switches 117a and 117b have a function for switching the connection destination of the electrodes 111a and 111b based on whether the electrocardiogram measurement unit 110 is performing human body communication. Specifically, in the example illustrated in FIG. 8, the switches 117a and 117b switch the connection destination of the electrodes 111a and 111b to the HBC reception unit 120 or the differential amplifier 112. It is noted that the switching by the switches 117a and 117b of the connection destination of the electrodes 111a and 111b may be performed by the HBC reception unit 120.

For example, when measuring the electrocardiogram waveform, the switches 117a and 117b switch the connection destination so that the electrodes 111a and 111b are connected to the differential amplifier 112, which is a subsequent-stage circuit in the electrocardiogram measurement unit 110. Further, for example, when various information transmitted from a pulse wave information measurement apparatus 20 is received by the electrocardiogram information measurement apparatus 10 by human body communication, the switches 117a and 117b switch the connection destination so that the electrodes 111a and 111b are connected to the HBC reception unit 120. Thus, during reception of the information, by configuring so that the electrodes 111a and 111b are not connected with the subsequent-stage differential amplifier 112, mixing of the received information in the electrocardiogram waveform result can be prevented. Further, since the electrocardiogram measurement is carried out at a high impedance, by switching the destination of the electrodes 111a and 111b based on whether electrocardiogram measurement is performed or whether human body communication is performed, a decline in impedance during the electrocardiogram measurement can be prevented, which enables the electrocardiogram measurement to be carried out more accurately.

In the above, a configuration example of the electrocardiogram measurement unit 110 was described. It is noted that the configuration of the electrocardiogram measurement unit 110 is not limited to the configuration illustrated in the FIG. 8. The electrocardiogram measurement unit 110 can have any circuit configuration, as long as it is capable of measuring the electrocardiogram waveform of the measurement subject.

The HBC reception unit 120 is a module for receiving data based on human body communication. The HBC reception unit 120 is an example of the biological information reception unit described above in “3.1. Pulse wave transit time calculation method”. In the first embodiment of the present disclosure, the HBC reception unit 120 receives the pulse wave detection packet 710 transmitted from the pulse wave information measurement apparatus in the manner described above with reference to FIG. 6.

When the HBC reception unit 120 is connected to the electrodes 111a and 111b of the electrocardiogram measurement unit 110, and is performing human body communication, the HBC reception unit 120 receives data via the electrodes 111a and 111b. Namely, in the present embodiment, the electrodes 111a and 111b have both a role of measuring the electrocardiogram waveform and a role of receiving data by human body communication. Thus, by combining the electrodes for measuring the electrocardiogram waveform and the electrodes for receiving data by human body communication in the electrodes 111a and 111b, the number of structures added to the electrocardiogram information measurement apparatus 10 can be comparatively reduced, and consequently an electrocardiogram information measurement apparatus can be realized that is more compact and has better portability.

It is noted that the frequency that is used for data transmission in human body communication is around 30 mHz. On the other hand, the heart rate and the pulse wave of a person have a period of about 1 s. Therefore, the frequency of the first waveform and the second waveform is in a very different band from the frequency of the data transmission by human body communication. Accordingly, as described above, the respective signals obtained by appropriately performing filter processing and the like can be distinguished from each other even if the electrocardiogram measurement and human body communication use the same electrodes, so that there is no signal mixing of the two.

The input terminal of the HBC reception unit 120 is a high impedance differential input, and is designed so that the input impedance does not decline during measurement of the electrocardiogram waveform. Further, as described above in “3.1. Pulse wave transit time calculation method”, under the control of the control unit 150, the HBC reception unit 120 may be started up for just the duration of the reception window 730 illustrated in FIG. 6. By setting the reception window 730, power consumption can be reduced and the probability of receiving an error can be decreased. The reason for this is because when the pulse wave detection packet 710 is transmitted at a timing that is not within the reception window 730, the timing when the initial rise point of the pulse wave is detected is substantially different from the timing that would be expected from the normal pulse wave transit time of the measurement subject.

The communication unit 130 is a communication interface for enabling the electrocardiogram information measurement apparatus 10 and an arbitrary external device to communicate with each other. For example, as described with reference to FIG. 3, the electrocardiogram information measurement apparatus 10 transmits information relating to the pulse wave transit time to the mobile terminal 690, which is an external device. As the communication method by the communication unit 130, for example, a wireless communication method, such as Bluetooth®, is used. However, the communication method by the communication unit 130 is not limited to this example. Any known communication method, regardless of whether it is wired or wireless, may be used.

The power unit 140, which is a power supply unit that includes a battery, supplies power to each of the constituent parts of the electrocardiogram information measurement apparatus 10. In order to reduce the size and weight of the electrocardiogram information measurement apparatus 10, for example, a coin type battery or the like is used for the battery in the power unit 140. Here, the destination that the power unit 140 supplies power to may be switched under the control of the control unit 150. For example, the power unit 140 may start up the HBC reception unit 120 by supplying power to the HBC reception unit 120 for just the period corresponding to the reception window 730 illustrated in FIG. 6.

The control unit 150 controls the electrocardiogram information measurement apparatus 10 in an integrated manner, and processes various data acquired by the electrocardiogram information measurement apparatus 10. Specifically, the control unit 150 performs processing to detect a first feature, which is a characteristic feature of the electrocardiogram waveform, based on electrocardiogram information relating to the measured electrocardiogram waveform of the measurement subject. Further, the control unit 150 starts up the HBC reception unit 120 for just a pulse wave information reception period, which is a predetermined duration, and during this pulse wave information reception period, performs processing for receiving the pulse wave information transmitted from the pulse wave information measurement apparatus. In addition, the control unit 150 performs processing for calculating the pulse wave transit time of the measurement subject based on electrocardiogram information and the received pulse wave information. In the following, the configuration of the control unit 150 will be described in more detail.

The control unit 150 includes a biological information acquisition unit 151, a first feature detection unit 152, a reception control unit 153, a power control unit 154, and a pulse wave transit time calculation unit 155.

The biological information acquisition unit 151 acquires biological information relating to the biological activity of the measurement subject. Here, in the following description, this biological information may be any information relating to the biological activity of the measurement subject. Examples of the biological information include information relating to an electrocardiograph (an electrocardiogram), pulse, heart rate, heart sound, breathing, body temperature and the like.

In the first embodiment of the present disclosure, as biological information, the biological information acquisition unit 151 acquires first waveform information relating to a first waveform representing the measurement subject's beat that was measured at a first measurement site. This first waveform information may be, specifically, electrocardiogram information relating to the electrocardiogram waveform measured at the measurement subject's chest. Further, in the present embodiment, as biological information, the biological information acquisition unit 151 acquires via the HBC reception unit 120 second waveform information relating to a second waveform representing the measurement subject's pulse that was measured at a second measurement site. This second waveform information may be, specifically, pulse wave information relating to the pulse wave measured at a finger on the measurement subject's hand by a pulse wave information measurement apparatus. The biological information acquisition unit 151 transmits the acquired electrocardiogram information to the first feature detection unit 152. Further, the biological information acquisition unit 151 transmits the acquired pulse wave information to the pulse wave transit time calculation unit 155.

The first feature detection unit 152 detects a first feature, which is a characteristic feature of the electrocardiogram waveform, based on the electrocardiogram information. Here, the first feature may be, for example, the initial rise or initial fall of a P wave, a Q wave, an R wave, an S wave, or a T wave included in the electrocardiogram waveform. However, the first feature is not limited to this example, and may be some other point in the electrocardiogram waveform. In the first embodiment of the present disclosure, the first feature detection unit 152 detects the initial rise point of the R wave in the electrocardiogram waveform as the first feature. The first feature detection unit 152 transmits information relating to the detected initial rise point of the R wave to the reception control unit 153 and the pulse wave transit time calculation unit 155.

The reception control unit 153 controls the HBC reception unit 120 so that the various information transmitted from the pulse wave information measurement apparatus is received by human body communication. Specifically, the reception control unit 153 controls the HBC reception unit 120 so that the pulse wave information transmitted from the pulse wave information measurement apparatus is received by human body communication. Here, as described above, rather than receiving all of the information relating to the pulse wave (the waveform data itself), information relating to the time corresponding to the second feature (e.g., an initial rise point), which is a characteristic feature of the pulse wave, is received as the pulse wave information.

Further, the reception control unit 153 can start up the HBC reception unit 120 for just a pulse wave information reception period, which is a predetermined duration, and during this pulse wave information reception period, control the control unit 150 so as to receive the pulse wave information transmitted from the pulse wave information measurement apparatus. Further, the pulse wave information reception period can be set based on a timing corresponding to the first feature detected by the first feature detection unit 152. For example, the reception control unit 153 can start up the HBC reception unit 120 after a predetermined duration has elapsed since the timing corresponding to the first feature, and switch the HBC reception unit 120 back to a sleep state after a predetermined duration has elapsed since the HBC reception unit 120 was started up.

The power control unit 154 controls the supply of power to each of the constituent parts of the electrocardiogram information measurement apparatus 10 by controlling the power unit 140. Here, the power control unit 154 can switch the destination that power is supplied to by the power unit 140. For example, if the reception control unit 153 starts up the HBC reception unit 120 for just a pulse wave information reception period, the power control unit 154 can control the supply of power to the HBC reception unit 120 to match that pulse wave information reception period.

The pulse wave transit time calculation unit 155 calculates the pulse wave transit time, which is the difference between the timing corresponding to the first feature and the timing corresponding to the second feature, based on the electrocardiogram information and the pulse wave information. In the example illustrated in FIG. 8, the pulse wave transit time calculation unit 155 calculates the pulse wave transit time by receiving the information relating to the first feature (the initial rise point of the R wave in the electrocardiogram waveform) from the first feature detection unit 152 and the information relating to the second feature (the initial rise point of the pulse wave) from the biological information acquisition unit 151 via the HBC reception unit 120. The information relating to the pulse wave transit time calculated by the pulse wave transit time calculation unit 155 is transmitted to an arbitrary external device via the communication unit 130, and the blood pressure value of the measurement subject is calculated by that external device based on the pulse wave transit time.

### (3.2.2. Pulse Wave Information Measurement Apparatus)

Next, the configuration of the pulse wave information measurement apparatus according to the first embodiment of the present disclosure will be described with reference to FIG. 9. As illustrated in FIG. 9, the pulse wave information measurement apparatus 20 according to the first embodiment of the present disclosure includes a pulse wave measurement unit 210, an HBC transmission unit 220, a power unit 230, and a control unit 240.

The pulse wave measurement unit 210 measures the pulse wave of the measurement subject at a second measurement site of the measurement subject. Specifically, the pulse wave measurement unit 210, which is worn on a finger on the measurement subject's hand, measures the pulse wave of the measurement subject with a below-described optical sensing unit 211. The pulse wave measurement unit 210 transmits data about the measured pulse wave to a below-described biological information acquisition unit 241 in the control unit 240.

Here, the specific configuration of the pulse wave measurement unit 210 will be described. The pulse wave measurement unit 210 includes an optical sensing unit 211, an amplifier 215, a band-pass filter 216, and an AD converter 217.

The optical sensing unit 211 performs an optical measurement for measuring the pulse wave at a pulse wave detection site. The optical sensing unit 211 is configured from a light-emitting element 212, a light-receiving element 213, and a sensing drive unit 214.

The light-emitting element 212 may be, for example, an LED that irradiates infrared light. It is noted that the wavelength of this infrared light may be about 940 nm, for example. The light-emitting element 212 is driven by the sensing drive unit 214 to irradiate light on the pulse wave detection site.

The light-receiving element 213, which is, for example, a photodiode, detects light that has passed through or was reflected by the pulse wave detection site of the light irradiated from the light-emitting element 212, and inputs a signal based on the received light amount to the amplifier 215. Here, for example, if the light-receiving element 213 detects light that has passed through the pulse wave detection site, the light-emitting element 212 and the light-receiving element 213 are arranged so as to sandwich the pulse wave detection site. Further, for example, if the light-receiving element 213 detects light that was reflected by the pulse wave detection site, the light-emitting element 212 and the light-receiving element 213 are arranged on the same side with respect to the pulse wave detection site.

The sensing drive unit 214 controls the drive of the light-emitting element 212 under the control of the control unit 240. In the present embodiment, the sensing drive unit 214 irradiates light having a predetermined wavelength on the pulse wave detection site by driving the light-emitting element 212 for the duration of the series of processes for calculating the pulse wave transit time.

The amplifier 215 amplifies an electric signal input from the light-receiving element 213, and outputs the amplified signal to a subsequent-stage band-pass filter 216. Here, the gain by the amplifier 215 may be appropriately set based on the light amount and the like of the light-emitting element 212.

The band-pass filter 216 removes unwanted noise from the signal input from the amplifier 215. The band-pass filter 216 is set based on the band of the light irradiated from the light-emitting element 212. For example, if the light-emitting element 212 irradiates infrared light, the band-pass filter 216 is set so as to reduce the frequency components other than the band corresponding to infrared light.

The signal in which noise has been reduced by passing through the band-pass filter 216 is input to the AD converter 217. Since the function of the AD converter 217 is the same as that of the AD converter 116, a detailed description of its functions will be omitted here. The signal digitalized by the AD converter 217 forms a pulse wave signal of the measurement subject. The AD converter 217 transmits the digitally-converted signal to the biological information acquisition unit 241 in the control unit 240.

In the above, a configuration example of the pulse wave measurement unit 210 was described. It is noted that the configuration of the pulse wave measurement unit 210 is not limited to the configuration illustrated in the FIG. 9. The pulse wave measurement unit 210 can have any circuit configuration, as long as it is capable of measuring the pulse wave of the measurement subject.

The HBC transmission unit 220 is a module for transmitting data based on human body communication. The HBC transmission unit 220 is an example of the biological information transmission unit described above in “3.1. Pulse wave transit time calculation method”. In the present embodiment, the HBC transmission unit 220 transmits the pulse wave detection packet 710 as pulse wave information in the manner described above with reference to FIG. 6. Further, as described above in “3.1. Pulse wave transit time calculation method”, transmission may be controlled by the control unit 240 so that the HBC transmission unit 220 is started up for just the period that the pulse wave detection packet 710 is being transmitted, and at other times the HBC transmission unit 220 is in a sleep state.

Further, the HBC transmission unit 220 has electrodes 221a and 221b for human body communication. The HBC transmission unit 220 transmits various types of data by human body communication by bringing the electrodes 221a and 221b into contact with the human body. Specifically, during data transmission, one of the electrodes 221a and 221b is used as a transmission output terminal, and the other is used as a ground. However, rather than using one of them as a ground, the electrodes 221a and 221b may both be used as balanced output terminals.

The power unit 230, which is a power supply unit that includes a battery, supplies power to each of the constituent parts of the pulse wave information measurement apparatus 20. In order to reduce the size and weight of the pulse wave information measurement apparatus 20, for example, a coin type battery or the like is used for the battery in the control unit 240. Here, the destination that the power unit 230 supplies power to may be switched under the control of the control unit 240. For example, the power unit 230 may start up the HBC transmission unit 220 by supplying power to the HBC transmission unit 220 for just the period that the pulse wave detection packet 710 is being transmitted.

The control unit 240 controls the pulse wave information measurement apparatus 20 in an integrated manner, and processes various data acquired by the pulse wave information measurement apparatus 20. Specifically, the control unit 240 performs processing to detect the second feature, which is a characteristic feature of the pulse wave, based on pulse wave information relating to the measured pulse wave of the measurement subject. Further, the control unit 240 controls the HBC transmission unit 220 and performs processing to transmit the pulse wave information to the electrocardiogram information measurement apparatus 10. In the following, the configuration of the control unit 240 will be described in more detail.

The control unit 240 includes a biological information acquisition unit 241, a second feature detection unit 242, a transmission control unit 243, and a power control unit 244.

Similar to the biological information acquisition unit 151 in the electrocardiogram information measurement apparatus 10, the biological information acquisition unit 241 acquires biological information relating to the biological activity of the measurement subject. In the first embodiment of the present disclosure, as biological information, the biological information acquisition unit 241 acquires second waveform information relating to a second waveform representing the measurement subject's beat that was measured at a second measurement site. This second waveform information may be, specifically, pulse wave information relating to the pulse wave measured at a finger on the measurement subject's hand by the pulse wave measurement unit 210. The biological information acquisition unit 241 transmits the acquired pulse wave information to the second feature detection unit 242.

The second feature detection unit 242 detects the second feature, which is a characteristic feature of the pulse wave, based on the pulse wave information. Here, the second feature may be, for example, the initial rise of the pulse wave. However, the second feature is not limited to this example, and may be some other point in the pulse wave. It is noted that the second feature detection unit 242 can detect the initial rise point by differentiating the pulse wave with respect to time twice, as described in “3.1. Pulse wave transit time calculation method”. The second feature detection unit 242 transmits information relating to the detected initial rise point to the transmission control unit 243.

The transmission control unit 243 controls the HBC transmission unit 220 so that various pieces of information are transmitted to the electrocardiogram information measurement apparatus 10 by human body communication. Specifically, the transmission control unit 243 controls the HBC transmission unit 220 so that the pulse wave detection packet 710 is transmitted as pulse wave information to the electrocardiogram information measurement apparatus 10 by human body communication. Further, the HBC transmission unit 220 may be controlled so that the HBC transmission unit 220 is started up for just the period that the pulse wave detection packet 710 is being transmitted, and at other times the HBC transmission unit 220 is in a sleep state.

The power control unit 244 controls the supply of power to the each of the constituent parts of the pulse wave information measurement apparatus 20 under the control of the power unit 230. Here, the power control unit 244 may switch the supply destination of power by the power unit 230. For example, if the transmission control unit 243 starts up the HBC transmission unit 220 for just the period that the pulse wave detection packet 710 is being transmitted, the power control unit 244 may control the supply of power to the HBC transmission unit 220 to match that start-up timing.

In the above, the configuration of the electrocardiogram information measurement apparatus 10 and the pulse wave information measurement apparatus 20 according to the first embodiment of the present disclosure was described with reference to FIGS. 8 and 9. Next, the various processing steps performed by the above-described electrocardiogram information measurement apparatus 10 and the pulse wave information measurement apparatus 20 will be described with reference to a sequence diagram.

### (3.3. Pulse Wave Transit Time Measurement Sequence)

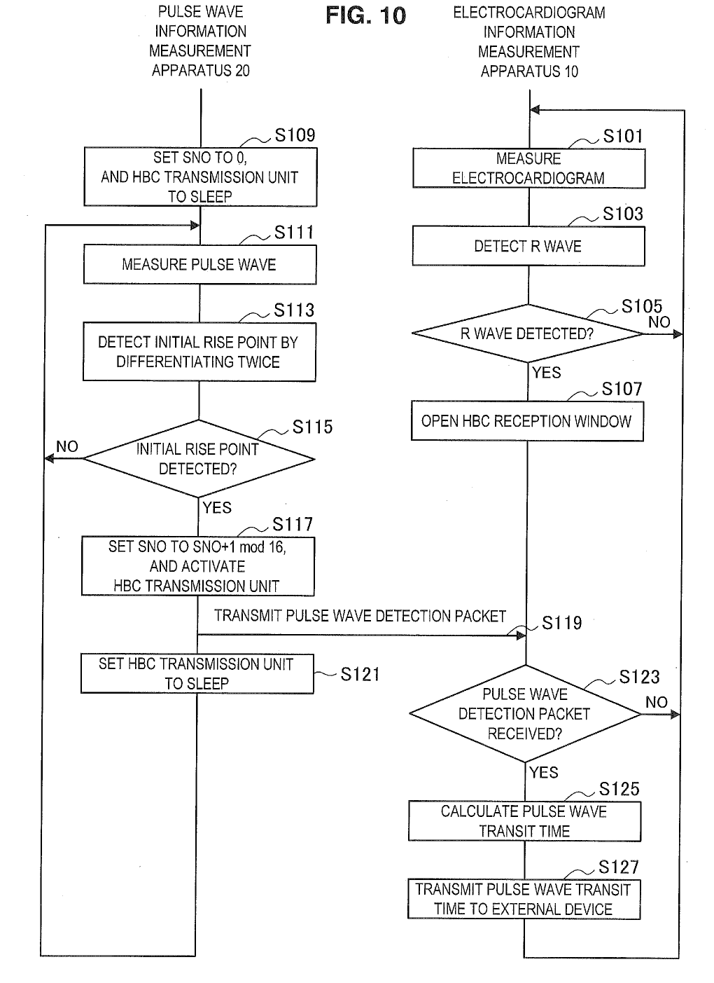

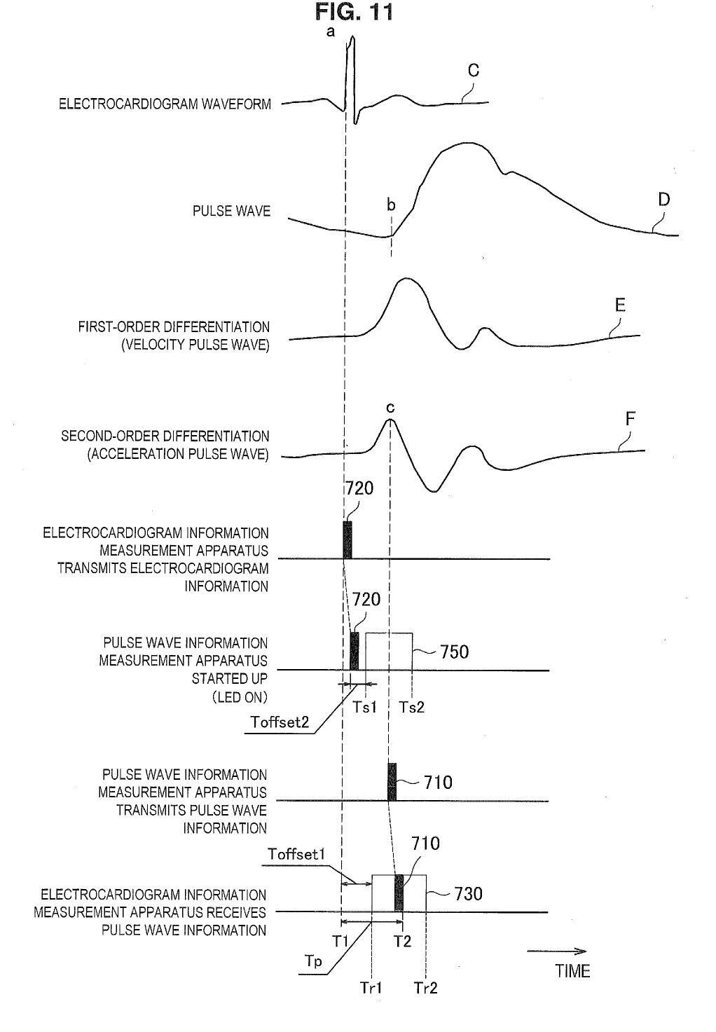

A pulse wave transit time calculation method according to the first embodiment of the present disclosure will now be described with reference to FIG. 10. FIG. 10 is a sequence diagram illustrating a pulse wave transit time calculation method according to the first embodiment of the present disclosure.

First, an electrocardiogram of the measurement subject is measured by the electrocardiogram information measurement apparatus 10 (step S101). Specifically, an electrocardiogram waveform of the measurement subject is measured by the electrocardiogram measurement unit 110 in the electrocardiogram information measurement apparatus 10.

Next, an R wave is detected from the measured electrocardiogram waveform (step S103). Specifically, the initial rise point of an R wave in the electrocardiogram waveform is detected by the first feature detection unit 152.

Next, in step S105, it is determined whether an R wave was detected from the electrocardiogram waveform. If an R wave was not detected from the electrocardiogram waveform, the processing returns to step S101, and electrocardiogram measurement is carried out again.

If an R wave was detected from the electrocardiogram waveform, the processing proceeds to step S107. In step S107, the HBC reception unit 120 is started up. However, in step S 107, the HBC reception unit 120 is started up just for a pulse wave information reception period, which is a predetermined duration. The pulse wave information reception period may be set in advance. For example, the pulse wave information reception period may be a period corresponding to the reception window 730 illustrated in FIG. 6.

On the other hand, parallel to the above-described steps S101 to 107, the processing of the following steps S109 to S119 is performed by the pulse wave information measurement apparatus 20.

First, at the pulse wave information measurement apparatus 20, before pulse wave measurement, in step S 109, processing to initialize the sequence number (SNO) 714 of the pulse wave detection packet to SNO=0 is performed. Further, at this stage, the HBC transmission unit 220 is in a sleep state.

Next, the pulse wave of the measurement subject is measured (step S111). Specifically, the pulse wave of the measurement subject is measured by the pulse wave measurement unit 210 in the pulse wave information measurement apparatus 20.

Next, the initial rise point is detected from the measured pulse wave (step S113). Specifically, the second feature detection unit 242 detects the initial rise point by differentiating the pulse wave with respect to time twice.

Next, in step S115, it is determined whether an initial rise point was detected from the pulse wave. If an initial rise point was not detected from the pulse wave, the processing returns to step S111, and pulse wave measurement is carried out again.

If an initial rise point was detected, the processing proceeds to step S117. In step S117, the HBC transmission unit 220 is started up, and one is added to the sequence number (SNO) 714 of the pulse wave detection packet up to an upper limit of 16. Then, in step S119, the pulse wave detection packet 710 from the started-up HBC transmission unit 220 is transmitted to the HBC reception unit 120 in the electrocardiogram information measurement apparatus 10. When the pulse wave detection packet 710 has been transmitted, the HBC transmission unit 220 switches to a sleep state (step S121).

Next, the following processing of steps S123 to S127 is performed at the electrocardiogram information measurement apparatus 10 to which the pulse wave detection packet 710 was transmitted.

In the electrocardiogram information measurement apparatus 10, since the HBC reception unit 120 has been started up in step S107 for a period corresponding to the reception window 730, the HBC reception unit 120 can receive the pulse wave detection packet 710 transmitted from the HBC transmission unit 220. When the pulse wave detection packet 710 transmitted in step S119 has been received, the electrocardiogram information measurement apparatus 10 determines whether the pulse wave detection packet 710 was properly received (step S123). Whether the pulse wave detection packet 710 was properly received can be determined based on, for example, error detection by the error detection code (CRC) 715 of the pulse wave detection packet 710, or based on gaps in the sequence number (SNO) 714.

If the pulse wave detection packet 710 was not properly received, the electrocardiogram information measurement apparatus 10 discards that pulse wave detection packet 710, the processing returns to step S101, and the series of steps for calculating the pulse wave transit time is carried out again.

If the pulse wave detection packet 710 was properly received, the pulse wave transit time, which is the difference between the timing corresponding to the first feature and the timing corresponding to the second feature, is calculated based on the electrocardiogram information and the pulse wave information (step S125). Specifically, the timing corresponding to the first feature may be the timing corresponding to the initial rise point of the R wave in the electrocardiogram waveform detected in step S103, and the timing corresponding to the second feature may be the timing corresponding to the initial rise point of the pulse wave detected in step S113.

After the pulse wave transit time has been detected, in step S127, information relating to this pulse wave transit time is transmitted to an arbitrary external device. Then, the measurement subject's blood pressure is calculated by that external device based on the pulse wave transit time.

By repeating the above-described processing performed in steps S101 to S127, the pulse wave transit time and blood pressure of the measurement subject can be constantly continuously measured.

In the above, the first embodiment of the present disclosure was described with reference to FIGS. 6, 7, 8, 9, and 10. As described above, in the first embodiment of the present disclosure, the electrocardiogram information measurement apparatus 10 measures the electrocardiogram waveform of the measurement subject, and the pulse wave information measurement apparatus 20 measures the pulse wave of the measurement subject. Further, the pulse wave detection packet 710 is transmitted from the pulse wave information measurement apparatus 20 to the electrocardiogram information measurement apparatus 10 as information relating to the pulse wave, and the pulse wave transit time is calculated based on electrocardiogram information relating to the electrocardiogram waveform and the pulse wave information. Thus, by transmitting and receiving only information relating to the timing corresponding to the second feature, which is a characteristic feature of the pulse wave, as pulse wave information rather than all the information relating to the pulse wave (the waveform data itself), the amount of data that is handled can be reduced, and a decrease in power consumption of the electrocardiogram information measurement apparatus 10 and the pulse wave information measurement apparatus 20 can be realized. Further, by reducing power consumption, the battery mounted in the electrocardiogram information measurement apparatus 10 and the pulse wave information measurement apparatus 20 can be made more compact, so that even better portability is achieved, and user friendliness for the measurement subject is improved.

Further, in the first embodiment of the present disclosure, the HBC reception unit 120 in the electrocardiogram information measurement apparatus 10 and the HBC transmission unit 220 in the pulse wave information measurement apparatus 20 are started up at a timing when data is transmitted from the pulse wave information measurement apparatus 20 to the electrocardiogram information measurement apparatus 10. Thus, by limiting the running time of the HBC reception unit 120 and the HBC transmission unit 220, the power consumption of the electrocardiogram information measurement apparatus 10 and the pulse wave information measurement apparatus 20 can be reduced even further.

In addition, in the first embodiment of the present disclosure, the transmission of data between the electrocardiogram information measurement apparatus 10 and the pulse wave information measurement apparatus 20 was performed using human body communication. Thus, by using human body communication that has a lower power consumption than other forms of wireless communication as the communication method between the two apparatuses, even further reductions in the power consumption of the electrocardiogram information measurement apparatus 10 and the pulse wave information measurement apparatus 20 are realized.

Further, in the first embodiment of the present disclosure, by thus using human body communication, a cable or other such connection between the electrocardiogram information measurement apparatus 10 and the pulse wave information measurement apparatus 20 does not have to be used. For example, when sleeping while wearing the electrocardiogram information measurement apparatus 10 and the pulse wave information measurement apparatus 20 in order to measure blood pressure while asleep, the presence of a cable or other such connection can hinder stable blood pressure measurement due to contact or interference with other objects caused by unintentional movements such as turning in bed. In the first embodiment of the present disclosure, since the use of human body communication makes it unnecessary to use a cable or other such connection, more stable blood pressure measurement is realized and user friendliness for the measurement subject is improved.

In addition, in the first embodiment of the present disclosure, in the electrocardiogram information measurement apparatus 10, the electrodes for human body communication are also used as the electrodes for electrocardiogram measurement. Therefore, the number of added structures for human body communication can be comparatively less, so that the electrocardiogram information measurement apparatus 10 can be more compact and have better portability.

### 4. SECOND EMBODIMENT OF THE PRESENT DISCLOSURE

Next, a second embodiment of the present disclosure will be described. In the second embodiment of the present disclosure, similar to the first embodiment of the present disclosure, an electrocardiogram sensor (electrocardiogram information measurement apparatus) and a pulse wave sensor (pulse wave information measurement apparatus) are configured as separate apparatuses. Further, in the second embodiment of the present disclosure, the apparatus having the function for calculating the pulse wave transit time is the electrocardiogram information measurement apparatus. The electrocardiogram information measurement apparatus calculates the pulse wave transit time based on pulse wave information transmitted from the pulse wave information measurement apparatus. However, the sequence when the pulse wave information measurement apparatus measures the pulse wave is different from in the first embodiment of the present disclosure. The following description of the second embodiment of the present disclosure will mainly be about the differences with the first embodiment of the present disclosure. A detailed description of overlapping functions and structures will be omitted here.

### (4.1. Pulse Wave Transit Time Calculation Method)

First, the method for calculating the pulse wave transit time according to the second embodiment of the present disclosure will be specifically described with reference to FIG. 11. FIG. 11 is an explanatory diagram illustrating a method for calculating the pulse wave transit time according to the second embodiment of the present disclosure. Here, the method for calculating the pulse wave transit time according to the second embodiment of the present disclosure will be described by comparing with FIG. 6, which illustrates the method for calculating the pulse wave transit time according to the first embodiment of the present disclosure.

As illustrated in FIG. 11, an electrocardiogram waveform C, a pulse wave D, a velocity pulse wave E, and an acceleration pulse wave F are on the same time axis. Further, in FIG. 11, the timing at which the pulse wave detection packet 710 is transmitted as pulse wave information from the pulse wave information measurement apparatus to the electrocardiogram information measurement apparatus is illustrated in association with the above waveforms. Since the waveforms and processing are the same as illustrated in FIG. 6, a detailed description will be omitted here.

In the second embodiment of the present disclosure, compared with the first embodiment of the present disclosure, as illustrated in FIG. 11, processing in which electrocardiogram information is transmitted from the electrocardiogram information measurement apparatus to the pulse wave information measurement apparatus, and processing in which pulse wave measurement is performed by the pulse wave information measurement apparatus for just a pulse wave measurement period (measurement window), which is a predetermined duration, are added. The following description of the method for calculating the pulse wave transit time will mainly be about these processes added to the second embodiment of the present disclosure.

In the second embodiment of the present disclosure, the electrocardiogram information measurement apparatus and the pulse wave information measurement apparatus, which both include a biological information transmission and reception unit, can transmit and receive various types of data between the two apparatuses.

For example, when a first feature is detected from the electrocardiogram waveform, the electrocardiogram information measurement apparatus transmits electrocardiogram information, which is information relating to the electrocardiogram waveform, to the pulse wave information measurement apparatus via the biological information transmission and reception unit. Here, in the second embodiment of the present disclosure, information relating to a time T1 corresponding to the initial rise point a of an R wave in an electrocardiogram waveform C is transmitted, rather than all the information relating to the electrocardiogram waveform C (the waveform data itself). In the example illustrated in FIG. 11, the electrocardiogram information measurement apparatus transmits to the pulse wave information measurement apparatus an R wave detection packet 720 as the electrocardiogram information. It is noted that the transmission of the R wave detection packet 720 from the electrocardiogram information measurement apparatus to the pulse wave information measurement apparatus is performed utilizing human body communication, for example. The R wave detection packet 720 is data in packet units indicating that the initial rise point a has been detected by the electrocardiogram information measurement apparatus from the electrocardiogram waveform C. Thus, by transmitting and receiving only information relating to the time corresponding to the initial rise point a of the electrocardiogram waveform C rather than all the information relating to the electrocardiogram waveform C, a decrease in power consumption of the electrocardiogram information measurement apparatus and the pulse wave information measurement apparatus can be realized. It is noted that since the configuration of the R wave detection packet 720 is the same as the configuration of the pulse wave detection packet 710 illustrated in FIG. 7, a detailed description thereof will be omitted here.

Further, in addition to the R wave detection packet 720, the electrocardiogram information measurement apparatus also transmits information relating to the pulse wave transit time measured the last time to the pulse wave information measurement apparatus via the biological information transmission and reception unit. The information relating to the pulse wave transit time is utilized when setting the pulse wave measurement period of the below-described pulse wave measurement unit in the pulse wave information measurement apparatus, for example.

Further, the electrocardiogram information measurement apparatus starts up the biological information transmission and reception unit for just the period that the R wave detection packet 720 and the information relating to the pulse wave transit time are being transmitted, and at other times switches the biological information transmission and reception unit to a sleep state. Namely, the electrocardiogram information measurement apparatus can start up the biological information transmission and reception unit for a limited time.

In addition, similar to when the pulse wave detection packet 710 is received by the electrocardiogram information measurement apparatus, the pulse wave information measurement apparatus can start up the biological information transmission and reception unit for just an electrocardiogram information reception period, which is a predetermined duration, when receiving the R wave detection packet 720 and the information relating to the pulse wave transit time. The reception window when receiving the R wave detection packet 720 can be set in a similar fashion to the reception window 730 for when the pulse wave detection packet 710 is received.

It is noted that, as illustrated in FIG. 11, a time lag caused by the time taken to transmit the R wave detection packet 720 and the time taken by the biological information transmission and reception unit to receive the R wave detection packet 720 is produced between the time corresponding to the actual initial rise point a of the R wave in the electrocardiogram waveform C and the time when the R wave detection packet 720 is received. However, as described above, since the configuration of the R wave detection packet 720 is the same as that of the pulse wave detection packet 710, as described regarding the pulse wave detection packet 710 in the first embodiment of the present disclosure, this time lag is about 500 μs. Therefore, considering the fact that pulse wave transit time is usually about 200 ms, similar to the pulse wave detection packet 710, the effect of this time lag caused by the transmission and reception of the R wave detection packet 720 on the ultimately-calculated blood pressure value can be ignored. It is noted that if the time lag can be predicted in advance, processing can be carried out to correct that time lag amount when calculating the pulse wave transit time.

In the second embodiment of the present disclosure, the pulse wave information measurement apparatus measures the pulse wave of the measurement subject only during the pulse wave transit time measurement period, which is a predetermined duration. In the example illustrated in FIG. 11, a case is illustrated in which, in the pulse wave information measurement apparatus, the below-described pulse wave measurement unit is started up during a period from time Ts1 to Ts2, and pulse wave measurement is carried out during this period. In the following description, this pulse wave measurement period, which is the time that the pulse wave measurement unit is started up, will also be referred to as a measurement window 750.

The time Ts1 that acts as a base point for the measurement window 750 and the time width (Ts2−Ts1) of the measurement window 750 are determined based on the timing T1 at which the R wave detection packet 720 transmitted from the electrocardiogram information measurement apparatus is received. Specifically, the value of the pulse wave transit time for the measurement subject is estimated based on a previous pulse wave transit time measurement value or based on a statistic obtained from past pulse wave transit time measurement values, for example.

Based on this predicted value, the center value of the measurement window 750 and the width of the measurement window 750 may be determined. This is possible because of the pulse wave's nature that when the measurement subject's pulse wave transit time is continuously measured, the pulse wave transit time does not greatly change. For example, as a specific example of a measurement window 750 setting, the measurement window 750 may be set by, based on a point that is past the timing at which the R wave detection packet 720 is received by the previous measurement value of the pulse wave transit time as the center value of the measurement window 750, providing a predetermined width from that center value of the measurement window 750. Namely, if the point that is past the timing at which the R wave detection packet 720 was received by the pulse wave information measurement apparatus by a duration of Toffset 2 is Ts1, then the measurement window 750 may be set as a relationship represented by Toffset 2=(previous pulse wave transit time measurement value)−(Ts2−Ts1). Alternatively, the measurement window 750 may be set as a point that is past Ts1 by a time width set based on the previous pulse wave transit time measurement value is Ts2. However, the time width of the pulse wave measurement period is set as a shorter time than the period of the pulse wave beat, including the timing corresponding to the initial rise point of the pulse wave. It is noted that the values of Ts1 and Ts2 may be appropriately set based on the individual differences of the measurement subject.

Thus, in the second embodiment of the present disclosure, processing for limiting the running time of the pulse wave measurement unit is added to the first embodiment of the present disclosure. Therefore, an even greater reduction in power consumption is realized for the pulse wave information measurement apparatus.

In the above, a method for calculating the pulse wave transit time according to the second embodiment of the present disclosure was described with reference to FIG. 11. Next, a specific configuration for realizing this method will be described.

### (4.2. Apparatus Configuration)

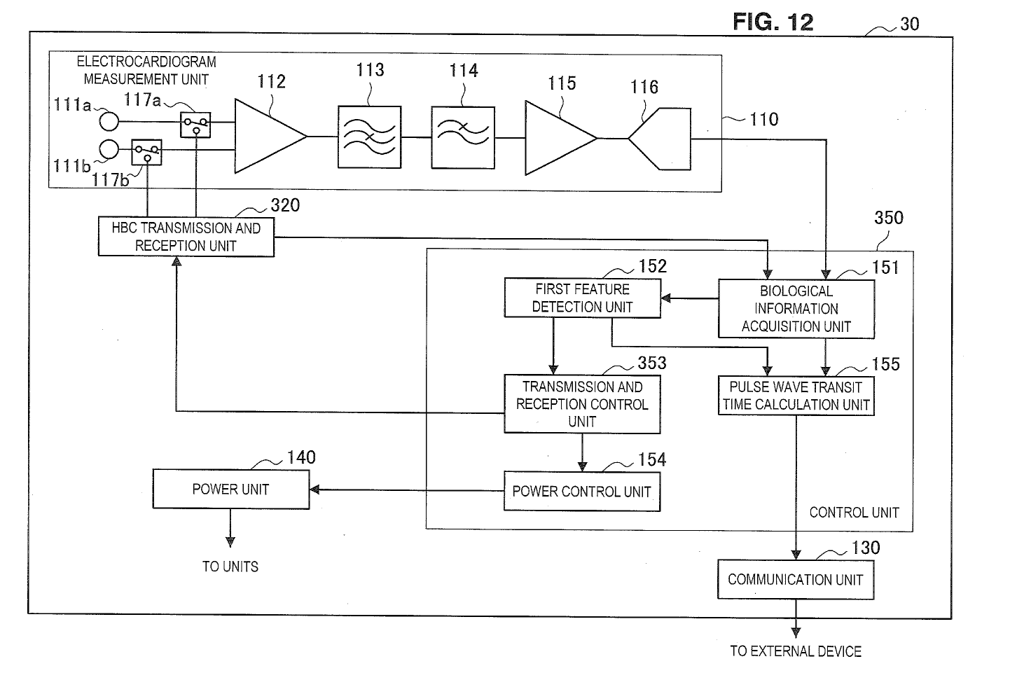

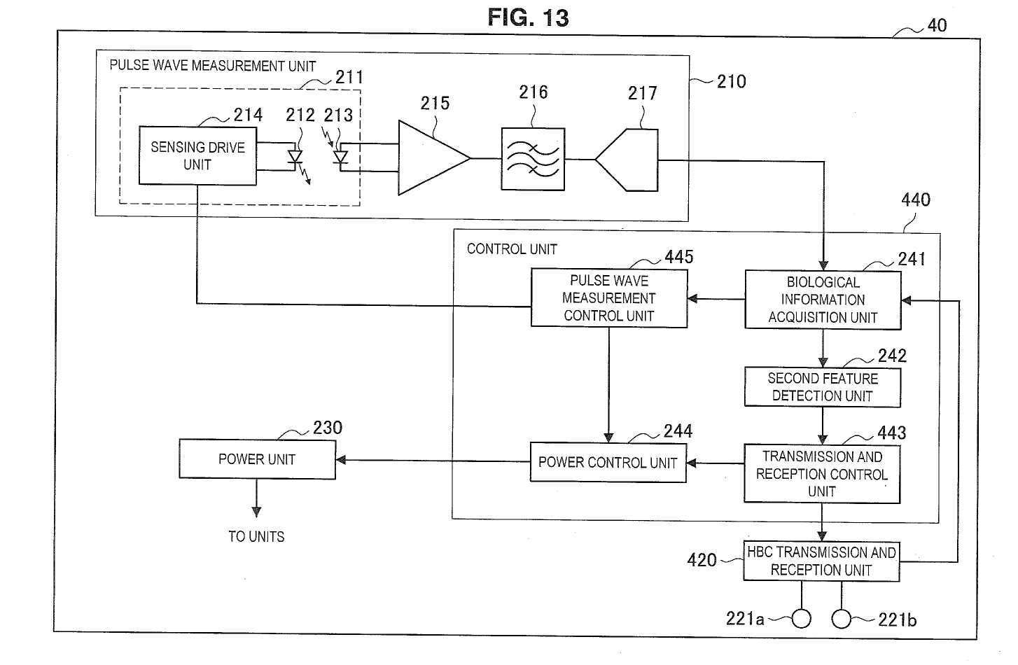

The configuration of the electrocardiogram information measurement apparatus and the pulse wave information measurement apparatus according to the second embodiment of the present disclosure will be specifically described with reference to FIGS. 12 and 13. FIG. 12 is a function block diagram illustrating a configuration example of the electrocardiogram information measurement apparatus according to the second embodiment of the present disclosure. Further, FIG. 13 is a function block diagram illustrating a configuration example of the pulse wave information measurement apparatus according to the second embodiment of the present disclosure.

### (4.2.1. Electrocardiogram Information Measurement Apparatus)

As illustrated in FIG. 12, an electrocardiogram information measurement apparatus 30 according to the second embodiment of the present disclosure includes an electrocardiogram measurement unit 110, an HBC transmission and reception unit 320, a communication unit 130, a power unit 140, and a control unit 350. Since the function and configuration of the electrocardiogram measurement unit 110, the communication unit 130, and the power unit 140 are the same as in the first embodiment of the present disclosure, a detailed description thereof will be omitted here.

The HBC transmission and reception unit 320 is a module for transmitting and receiving data based on human body communication. Although in the first embodiment of the present disclosure, the electrocardiogram information measurement apparatus 10 only receives various types of data by human body communication, in the second embodiment of the present disclosure, the electrocardiogram information measurement apparatus 30 can transmit and receive various types of data via the HBC transmission and reception unit 320. The HBC transmission and reception unit 320 is an example of the biological information transmission and reception unit described above in “4.2. Pulse wave transit time calculation method”. In the present embodiment, the HBC transmission and reception unit 320 transmits an R wave detection packet 720 and information relating to the pulse wave transit time to the pulse wave information measurement apparatus in the manner described above with reference to FIG. 11. Further, the HBC transmission and reception unit 320 receives a pulse wave detection packet 710 transmitted from the pulse wave information measurement apparatus. It is noted that other than the added data transmission function, the configuration of the HBC transmission and reception unit 320 may be the same as the HBC reception unit 120 in the electrocardiogram information measurement apparatus 10.

Although the function and the configuration of the electrocardiogram measurement unit 110 are the same as in the first embodiment of the present disclosure, in the second embodiment of the present disclosure, the switching by the switches 117a and 117b of the connection destination of the electrodes 111a and 111b may be performed by the HBC transmission and reception unit 320. For example, when measuring the electrocardiogram waveform, the switches 117a and 117b switch the connection destination so that the electrodes 111a and 111b are connected to the differential amplifier 112, which is a subsequent-stage circuit in the electrocardiogram measurement unit 110. Further, for example, when exchanging various information from the electrocardiogram information measurement apparatus 30 to a pulse wave information measurement apparatus 40 by human body communication, the switches 117a and 117b switch the connection destination so that the electrodes 111a and 111b are connected to the HBC transmission and reception unit 320. Thus, during transmission and reception of the information, by configuring so that the electrodes 111a and 111b are not connected with the subsequent-stage differential amplifier 112, mixing of the transmitted and received information in the electrocardiogram waveform result can be prevented. Further, similar to the first embodiment of the present disclosure, since the electrocardiogram measurement is carried out at a high impedance, by switching the destination of the electrodes 111a and 111b based on whether electrocardiogram measurement is performed or whether human body communication is performed, a decline in impedance during the electrocardiogram measurement can be prevented, which enables the electrocardiogram measurement to be carried out more accurately.

The control unit 350 controls the electrocardiogram information measurement apparatus 30 in an integrated manner, and processes various data acquired by the electrocardiogram information measurement apparatus 30. Specifically, the control unit 350 performs processing to detect a first feature, which is a characteristic feature of the electrocardiogram waveform, based on electrocardiogram information relating to the measured electrocardiogram waveform of the measurement subject. Further, the control unit 350 starts up the HBC transmission and reception unit 320 and performs processing for transmitting the electrocardiogram information and information relating to the pulse wave transit time to the pulse wave information measurement apparatus. In addition, the control unit 350 starts up the HBC transmission and reception unit 320 for just a pulse wave information reception period, which is a predetermined duration, and during this pulse wave information reception period, performs processing for receiving the pulse wave information transmitted from the pulse wave information measurement apparatus. In addition, the control unit 350 performs processing for calculating the pulse wave transit time of the measurement subject based on electrocardiogram information and the received pulse wave information. In the following, the configuration of the control unit 350 will be described in more detail.

The control unit 350 includes a biological information acquisition unit 151, a first feature detection unit 152, a transmission and reception control unit 353, a power control unit 154, and a pulse wave transit time calculation unit 155. Among the functions and structures of the control unit 350, since those of the biological information acquisition unit 151, the first feature detection unit 152, the power control unit 154, and the pulse wave transit time calculation unit 155 are the same as in the first embodiment of the present disclosure, a detailed description thereof will be omitted here.

The transmission and reception control unit 353 controls the HBC transmission and reception unit 320 so that various types of information are exchanged with the pulse wave information measurement apparatus via human body communication. Specifically, the transmission and reception control unit 353 controls the HBC transmission and reception unit 320 so that electrocardiogram information and information relating to the pulse wave transit time are transmitted to the pulse wave information measurement apparatus via human body communication. Further, the transmission and reception control unit 353 controls the HBC transmission and reception unit 320 so that pulse wave information transmitted from the pulse wave information measurement apparatus is received via human body communication. Here, as described above, rather than transmitting and receiving all of the information relating to the electrocardiogram waveform and the pulse wave (the waveform data itself), information relating to the timing corresponding to a first feature (e.g., initial rise point of the R wave) and the timing corresponding to a second feature (e.g., initial rise point), which are characteristic features of the electrocardiogram waveform and the pulse wave, are transmitted and received as the electrocardiogram information and the pulse wave information. It is noted that other than the control for transmitting the electrocardiogram information and the information relating to the pulse wave transit time to the pulse wave information measurement apparatus, the functions of the transmission and reception control unit 353 may be the same as those of the reception control unit 153 according to the first embodiment of the present disclosure.

It is also noted that, as described above, the HBC transmission and reception unit 320 may be started up just for the period when electrocardiogram information and information relating to the pulse wave transit time are being transmitted and when pulse wave information is being received. Thus, if the start-up of the HBC transmission and reception unit 320 is controlled, the power control unit 154 controls the power unit 140 so that power is supplied to the HBC transmission and reception unit 320 to match the start-up of the HBC transmission and reception unit 320.

### (4.2.2. Pulse Wave Information Measurement Apparatus)

Next, the configuration of the pulse wave information measurement apparatus according to the second embodiment of the present disclosure will be described with reference to FIG. 13. As illustrated in FIG. 13, the pulse wave information measurement apparatus 40 according to the second embodiment of the present disclosure includes a pulse wave measurement unit 210, an HBC transmission and reception unit 420, a power unit 230, and a control unit 440. Here, since the function and configuration of the pulse wave measurement unit 210 and the power unit 230 are the same as in the first embodiment of the present disclosure, a detailed description thereof will be omitted here.

The HBC transmission and reception unit 420 is a module for transmitting and receiving data based on human body communication. Although in the first embodiment of the present disclosure, the pulse wave information measurement apparatus 20 only transmits various types of data by human body communication, in the second embodiment of the present disclosure, the pulse wave information measurement apparatus 40 can transmit and receive various types of data via the HBC transmission and reception unit 420. The HBC transmission and reception unit 420 is an example of the biological information transmission and reception unit described above in “4.1. Pulse wave transit time calculation method”. In the present embodiment, the HBC transmission and reception unit 420 receives an R wave detection packet 720 and information relating to the pulse wave transit time that is transmitted from the electrocardiogram information measurement apparatus 30 in the manner described above with reference to FIG. 13. It is noted that the HBC transmission and reception unit 420 transmits the received R wave detection packet 720 and information relating to the pulse wave transit time to the biological information acquisition unit 241. Further, the HBC transmission and reception unit 420 transmits a pulse wave detection packet 710 to the electrocardiogram information measurement apparatus 30. It is noted that other than the added data reception function, the configuration of the HBC transmission and reception unit 420 may be the same as the HBC reception unit 120 in the pulse wave information measurement apparatus 20.

The control unit 440 controls the pulse wave information measurement apparatus 40 in an integrated manner, and processes various data acquired by the pulse wave information measurement apparatus 40. Specifically, the control unit 440 performs processing to detect a second feature, which is a characteristic feature of the pulse wave, based on pulse wave information relating to the measured pulse wave of the measurement subject. Further, the control unit 440 controls the HBC transmission and reception unit 440, and performs processing for receiving the R wave detection packet 720 and information relating to the pulse wave transit time transmitted from the electrocardiogram information measurement apparatus 30. In addition, the control unit 440 controls the HBC transmission unit 220 and performs processing to transmit the pulse wave information to the electrocardiogram information measurement apparatus 30. In the following, the configuration of the control unit 440 will be described in more detail.

The control unit 440 includes the biological information acquisition unit 241, the second feature detection unit 242, a transmission and reception control unit 443, a power control unit 244, and a pulse wave measurement control unit 445. Among the functions and structures of the control unit 440, since those of the biological information acquisition unit 241, the second feature detection unit 242, and the power control unit 244 are the same as in the first embodiment of the present disclosure, a detailed description thereof will be omitted here.

The transmission and reception control unit 443 controls the HBC transmission and reception unit 420 so that various types of information are exchanged with the electrocardiogram information measurement apparatus 30 via human body communication. Specifically, the transmission and reception control unit 443 controls the HBC transmission and reception unit 420 so that an R wave detection packet and information relating to the pulse wave transit time transmitted from the electrocardiogram information measurement apparatus 30 are received. Further, the transmission and reception control unit 443 controls the HBC transmission and reception unit 420 so that a pulse wave detection packet 710 is transmitted to the electrocardiogram information measurement apparatus 30 as pulse wave information. Further, the transmission and reception control unit 443 can control the HBC transmission and reception unit 420 so that the HBC transmission and reception unit 420 is started up for just the period that the R wave detection packet and the information relating to the pulse wave transit time are being received and the period that the pulse wave detection packet 710 is being transmitted, and at other times is in a sleep state. It is noted that other than the control for receiving the electrocardiogram information and the information relating to the pulse wave transit time, the functions of the transmission and reception control unit 443 may be the same as those of the transmission control unit 243 according to the first embodiment of the present disclosure.

It is also noted that, as described above, the HBC transmission and reception unit 420 may be started up just for the period when electrocardiogram information and information relating to the pulse wave transit time are being received and when pulse wave information is being transmitted. Thus, if the start-up of the HBC transmission and reception unit 420 is controlled, the power control unit 244 can control the power unit 430 so that power is supplied to the HBC transmission and reception unit 420 to match the start-up of the HBC transmission and reception unit 420.

The pulse wave measurement control unit 445 controls the drive of the pulse wave measurement unit 210 so as to measure the pulse wave of the measurement subject. Specifically, the pulse wave measurement control unit 445 controls the drive of the sensing drive unit 214 in the pulse wave measurement unit 210 so as to irradiate light on a second measurement site (pulse wave detection site) of the measurement subject from the light-emitting element 212 at a desired timing. Further, the pulse wave measurement control unit 445 can also control the drive of the light-emitting element 212 so that pulse wave measurement is carried out only during the pulse wave measurement period described with reference to FIG. 11. It is noted that to set the pulse wave measurement period, the pulse wave measurement control unit 445 can acquire from the biological information acquisition unit 241 the R wave detection packet 720 and information relating to the pulse wave transit time.

It is noted that, as described above, the pulse wave measurement unit 210 can be started up for just the pulse wave measurement period, which is a predetermined duration. If the start up of the pulse wave measurement unit 210 is limited in such a manner, the power control unit 244 controls the power unit 230 so that power is supplied to the pulse wave measurement unit 210 to match the start up of the pulse wave measurement unit 210.

In the above, the configuration of the electrocardiogram information measurement apparatus 30 and the pulse wave information measurement apparatus 40 according to the second embodiment of the present disclosure was described with reference to FIGS. 12 and 13. Next, the various processing steps performed by the above-described electrocardiogram information measurement apparatus 30 and the pulse wave information measurement apparatus 40 will be described with reference to a sequence diagram.

### (4.3. Pulse Wave Transit Time Measurement Sequence)

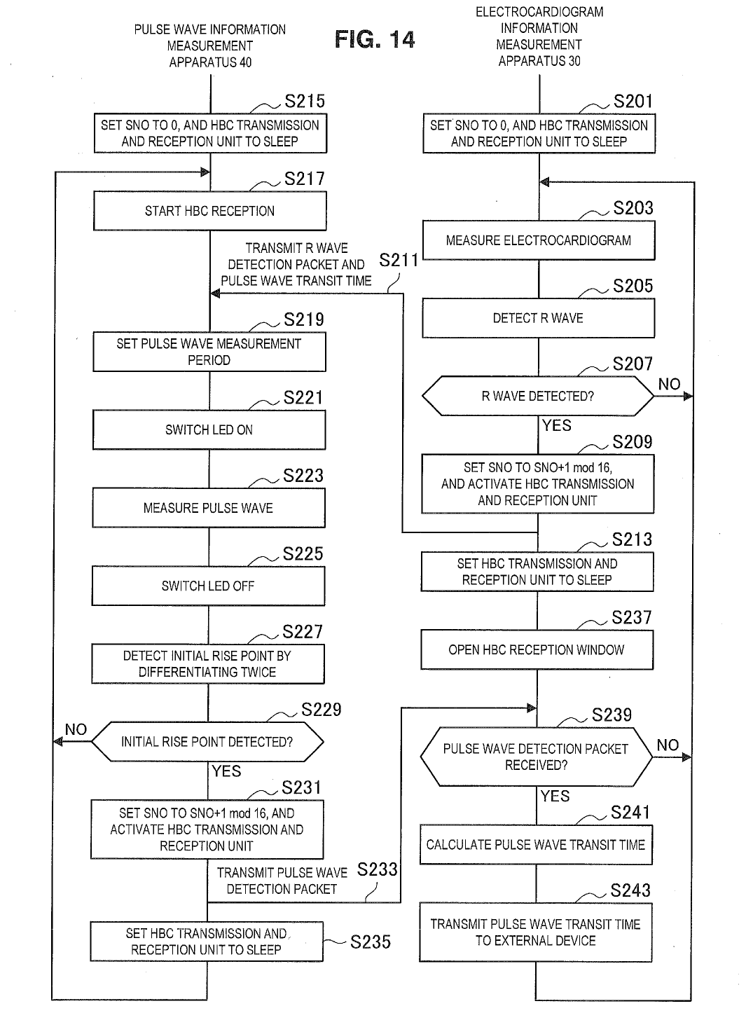

A pulse wave transit time calculation method according to the second embodiment of the present disclosure will now be described with reference to FIG. 14. FIG. 14 is a sequence diagram illustrating a pulse wave transit time calculation method according to the second embodiment of the present disclosure.

First, at the electrocardiogram information measurement apparatus 30, before electrocardiogram measurement, in step S201, processing to initialize the sequence number (SNO) of the R wave detection packet 720 to SNO=0 is performed. Further, at this stage, the HBC transmission and reception unit 320 is in a sleep state.

Next, an electrocardiogram of the measurement subject is measured by the electrocardiogram information measurement apparatus 30 (step S203). Specifically, an electrocardiogram waveform of the measurement subject is measured by the electrocardiogram measurement unit 110 in the electrocardiogram information measurement apparatus 30.

Next, an R wave is detected from the measured electrocardiogram waveform (step S205). Specifically, the initial rise point of an R wave in the electrocardiogram waveform is detected by the first feature detection unit 152.

Next, in step S207, it is determined whether an R wave was detected from the electrocardiogram waveform. If an R wave was not detected from the electrocardiogram waveform, the processing returns to step S203, and electrocardiogram measurement is carried out again.

If an R wave was detected from the electrocardiogram waveform, the processing proceeds to step S209. In step S209, the HBC transmission and reception unit 320 is started up, and one is added to the sequence number (SNO) of the R wave detection packet 720 up to an upper limit of 16. Then, in step S211, the R wave detection packet 720 from the started-up HBC transmission and reception unit 320 and information relating to the pulse wave transit time measured the last time are transmitted to the HBC transmission and reception unit 420 in the pulse wave information measurement apparatus 40. When the R wave detection packet 720 and information relating to the pulse wave transit time have been transmitted, the HBC transmission and reception unit 320 switches to a sleep state (step S213).

On the other hand, at the pulse wave information measurement apparatus 40, before receiving the R wave detection packet 720 and information relating to the pulse wave transit time, in step S215, processing to initialize the sequence number (SNO) 714 of the pulse wave detection packet 710 to SNO=0 is performed. Further, at this stage, the HBC transmission and reception unit 420 is in a sleep state. Further, at the pulse wave information measurement apparatus 40, before receiving the R wave detection packet 720 and information relating to the pulse wave transit time, the HBC transmission and reception unit 420 is started up (step S217). However, in step S217, the HBC transmission and reception unit 420 is started up for just an electrocardiogram information reception period, which is a predetermined duration. The electrocardiogram information reception period may be set in advance based on, for example, the method described above in “4.1. Pulse wave transit time calculation method”.

At the pulse wave information measurement apparatus 40 that has received the R wave detection packet 720 and information relating to the pulse wave transit time, a pulse wave measurement period is set based of these pieces of information (step S219). It is noted that pulse wave measurement period may be a period corresponding to the width of the measurement window 750 illustrated in FIG. 11, for example.

Next, at the pulse wave information measurement apparatus 40, the light-emitting element 212, for example an LED, in the pulse wave measurement unit 210 is driven (step S221). Then, the measurement subject's pulse wave is measured (step S223), and after a predetermined duration has elapsed, the drive of the LED is stopped (step S225). It is noted that the duration from after the LED is driven in step S221 until the LED is stopped being driven in step S225 is a time corresponding to the pulse wave measurement period set in step S219.

Next, the initial rise point is detected from the measured pulse wave (step S227). Specifically, the second feature detection unit 242 detects the initial rise point by differentiating the pulse wave with respect to time twice.

Next, in step S229, it is determined whether an initial rise point was detected from the pulse wave. If an initial rise point was not detected from the pulse wave, the processing returns to step S217, the R wave detection packet 720 and the information relating to the pulse wave transit time are received again, and the setting of the pulse wave measurement period and pulse wave measurement are carried out again.

If an initial rise point was detected, the processing proceeds to step S231. In step S231, the HBC transmission and reception unit 420 is started up, and one is added to the sequence number (SNO) 714 of the pulse wave detection packet 710 up to an upper limit of 16. Then, in step S233, the pulse wave detection packet 710 from the started-up HBC transmission and reception unit 420 is transmitted to the HBC transmission and reception unit 320 in the electrocardiogram information measurement apparatus 30. When the pulse wave detection packet 710 has been transmitted, the HBC transmission and reception unit 420 switches to a sleep state (step S235).

On the other hand, at the electrocardiogram information measurement apparatus 30, before receiving the pulse wave detection packet 710, the HBC transmission and reception unit 320 is started up (step S237). However, in step S237, the HBC transmission and reception unit 320 is started up just for a pulse wave information reception period, which is a predetermined duration. The pulse wave information reception period may be set in advance. For example, the pulse wave information reception period may be a period corresponding to the reception window 730 illustrated in FIG. 11.

Next, the following processing of steps S239 to S243 is performed at the electrocardiogram information measurement apparatus 30 to which the pulse wave detection packet 710 was transmitted.

At the electrocardiogram information measurement apparatus 30, since the HBC transmission and reception unit 320 has been started up in step S237 for a period corresponding to the reception window 730, the HBC transmission and reception unit 320 can receive the pulse wave detection packet 710 transmitted from the HBC transmission and reception unit 420 of the pulse wave information measurement apparatus 40. When the pulse wave detection packet 710 transmitted in step S233 has been received, the electrocardiogram information measurement apparatus 30 determines whether the pulse wave detection packet 710 was properly received (step S239). Whether the pulse wave detection packet 710 was properly received can be determined based on, for example, error detection by the error detection code (CRC) 715 of the pulse wave detection packet 710, or based on gaps in the sequence number (SNO) 714.

If the pulse wave detection packet 710 was not properly received, the electrocardiogram information measurement apparatus 30 discards that pulse wave detection packet 710, the processing returns to step S203, and the series of steps for calculating the pulse wave transit time is carried out again.

If the pulse wave detection packet 710 was properly received, the pulse wave transit time, which is the difference between the timing corresponding to the first feature and the timing corresponding to the second feature, is calculated based on the electrocardiogram information and the pulse wave information (step S241). Specifically, the timing corresponding to the first feature may be the timing corresponding to the initial rise point of the R wave in the electrocardiogram waveform detected in step S205, and the timing corresponding to the second feature may be the timing corresponding to the initial rise point of the pulse wave detected in step S227.

After the pulse wave transit time has been detected, in step S243, information relating to this pulse wave transit time is transmitted to an arbitrary external device. Then, the measurement subject's blood pressure is calculated by that external device based on the pulse wave transit time.

By repeating the above-described processing performed in steps S201 to S243, the pulse wave transit time and even the blood pressure of the measurement subject can be constantly continuously measured.

In the above, the second embodiment of the present disclosure was described with reference to FIGS. 11, 12, 13, and 14. In addition to the advantageous effects of the first embodiment of the present disclosure, in the second embodiment of the present disclosure the following advantageous effects can be obtained.

In the second embodiment of the present disclosure, the electrocardiogram information measurement apparatus 30 measures the electrocardiogram waveform of the measurement subject, and the pulse wave information measurement apparatus 40 starts up the pulse wave measurement unit for just the pulse wave measurement period, which is a predetermined duration, and measures the pulse wave of the measurement subject. Thus, by limiting the duration for measuring the pulse wave, the power consumed by the electrocardiogram information measurement apparatus 30 and the pulse wave information measurement apparatus 40 can be decreased compared with when the pulse wave is constantly measured.

Further, in the second embodiment of the present disclosure, the R wave detection packet 720 and the pulse wave transit time measured the last time are transmitted from the electrocardiogram information measurement apparatus 30 to the pulse wave information measurement apparatus 40, and the pulse wave detection packet 710 is transmitted from the pulse wave information measurement apparatus 40 to the electrocardiogram information measurement apparatus 30. Thus, by transmitting and receiving only information relating to the timing corresponding to the time corresponding to the first feature and the second feature, which are characteristic features of the electrocardiogram waveform and the pulse wave, as electrocardiogram information and the pulse wave information rather than all the information relating to the electrocardiogram waveform and the pulse wave (the waveform data itself), the amount of data that is handled can be reduced, and a decrease in power consumption of the electrocardiogram information measurement apparatus 30 and the pulse wave information measurement apparatus 40 can be realized.

Further, in the second embodiment of the present disclosure, the HBC transmission and reception unit 320 in the electrocardiogram information measurement apparatus 30 and the HBC transmission and reception unit 420 in the pulse wave information measurement apparatus 40 are started up at a timing when data is exchanged between the electrocardiogram information measurement apparatus 30 and the pulse wave information measurement apparatus 40. Thus, by limiting the running time of the HBC transmission and reception unit 320 and the HBC transmission and reception unit 420, the power consumption of the electrocardiogram information measurement apparatus 10 and the pulse wave information measurement apparatus 20 can be reduced even further.

### 5. THIRD EMBODIMENT OF THE PRESENT DISCLOSURE

Next, a third embodiment of the present disclosure will be described. In the third embodiment of the present disclosure, unlike the first embodiment or the second embodiment of the present disclosure, an electrocardiogram sensor and a pulse wave sensor are configured integrally, and are incorporated in a single apparatus (biological information measurement apparatus). Therefore, the sequence for calculating the pulse wave transit time is different from the first embodiment and the second embodiment of the present disclosure. The following description of the third embodiment of the present disclosure will mainly be about the differences with the first embodiment and the second embodiment of the present disclosure. A detailed description of overlapping functions and structures will be omitted here.

### (5.1. Pulse Wave Transit Time Calculation Method)

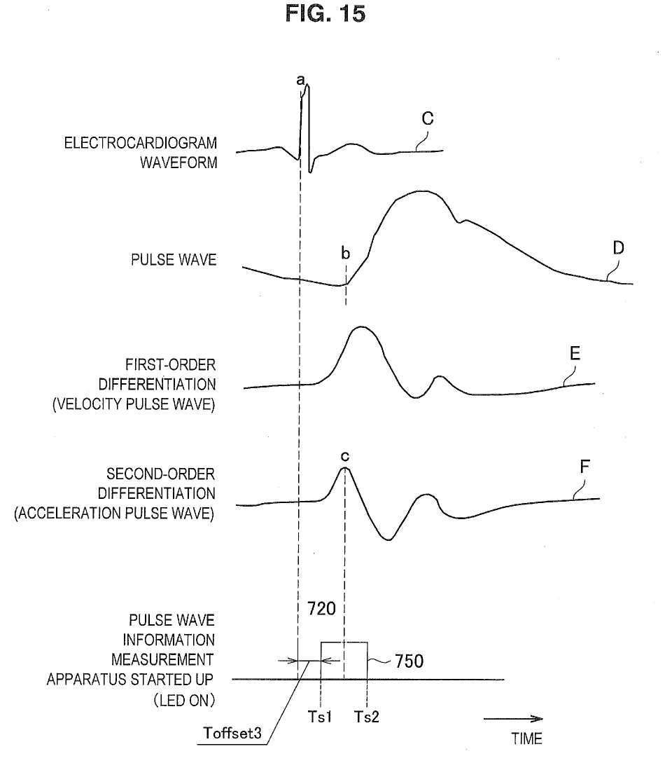

First, the method for calculating the pulse wave transit time according to the third embodiment of the present disclosure will be specifically described with reference to FIG. 15. FIG. 15 is an explanatory diagram illustrating a method for calculating the pulse wave transit time according to the third embodiment of the present disclosure. Here, the method for calculating the pulse wave transit time according to the third embodiment of the present disclosure will be described by comparing with FIG. 11, which illustrates the method for calculating the pulse wave transit time according to the second embodiment of the present disclosure.

As illustrated in FIG. 15, an electrocardiogram waveform C, a pulse wave D, a velocity pulse wave E, and an acceleration pulse wave F are on the same time axis. Since these waveforms are the same as in FIG. 11, a detailed description will be omitted here. Further, in FIG. 15, the timing at which the pulse wave measurement unit is started up is illustrated in association with the above waveforms.

As illustrated in FIG. 15, in the third embodiment of the present disclosure, similar to the second embodiment of the present disclosure, processing is performed in which the pulse wave sensor (pulse wave measurement unit) is started up for just a pulse wave measurement period, which is a predetermined duration. However, because the electrocardiogram sensor and the pulse wave sensor are integrally configured, the sequence of that processing is different from the second embodiment of the present disclosure. The following description will be mainly about this difference.

In the third embodiment of the present disclosure, as described above, the electrocardiogram sensor and the pulse wave sensor are integrally configured. Specifically, the biological information measurement apparatus according to the third embodiment of the present disclosure includes, for example, a ring-type pulse wave measurement unit and a patch-type electrocardiogram measurement unit. These two measurement units are connected with a cable or the like to form a single apparatus. It is noted that the control unit that controls the biological information measurement unit in an integrated manner may be mounted in a unit for pulse wave measurement included in a pulse wave measurement unit, or mounted in a unit for electrocardiogram measurement included in an electrocardiogram measurement unit.

First, the electrocardiogram measurement unit measures the measurement subject's electrocardiogram waveform C, and a first feature is detected by the control unit. In the example illustrated in FIG. 15, an initial rise point of an R wave in the electrocardiogram waveform C is detected as the first feature.

When an initial rise point a is detected, a pulse wave measurement period during which the pulse wave measurement unit is started up is set based on the timing T1 corresponding to the initial rise point a. In the example illustrated in FIG. 15, during the period from Ts1 to Ts2, the pulse wave measurement unit is started up. This duration that the pulse wave measurement unit is started up for corresponds to the pulse wave measurement period (the measurement window 750) according to the second embodiment of the present disclosure. Here, Ts1 is, for example, a point after T1 by a Toffset 3. The value of the time width (Ts2 0−Toffset 3) of the Toffset 3 and the pulse wave measurement period is set based on a previous pulse wave transit time measurement value or based on a statistic obtained from past pulse wave transit time measurement values. This is possible because of the pulse wave's nature that when the measurement subject's pulse wave transit time is continuously measured, the pulse wave transit time does not greatly change.

In the above, a method for calculating the pulse wave transit time according to the second embodiment of the present disclosure was described with reference to FIG. 15. Next, a specific configuration for realizing this method will be described.

### (5.2. Apparatus Configuration)

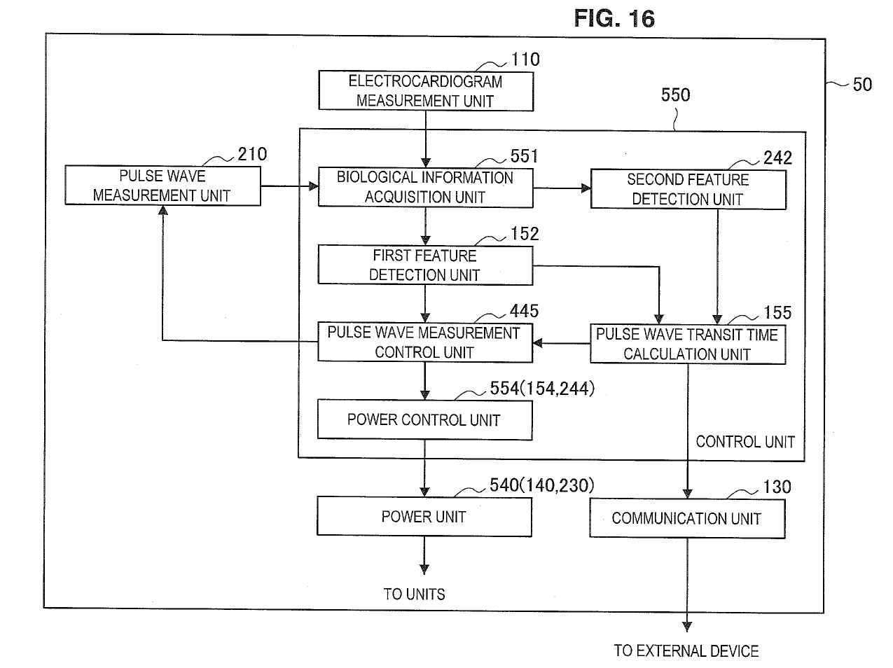

The configuration of the biological information measurement apparatus according to the third embodiment of the present disclosure will be specifically described with reference to FIG. 16. FIG. 16 is a function block diagram illustrating a configuration example of the biological information measurement apparatus according to the third embodiment of the present disclosure.

As illustrated in FIG. 16, a biological information measurement apparatus 50 according to the third embodiment of the present disclosure includes an electrocardiogram measurement unit 110, a pulse wave measurement unit 210, a communication unit 130, a power unit 540, and a control unit 550. Since the function and configuration of the electrocardiogram measurement unit 110, the pulse wave measurement unit 210, and the communication unit 130 are the same as in the first and second embodiments of the present disclosure, a detailed description thereof will be omitted here. Further, since the function and configuration of the power unit 540 are the same as those of the power unit 140 according to the first embodiment of the present disclosure and the power unit 230 according to the second embodiment of the present disclosure, a detailed description thereof will be omitted here. Therefore, the following description of the third embodiment of the present disclosure will mainly be about the function and configuration of the control unit 550.

The control unit 550 controls the biological information measurement apparatus 50 in an integrated manner, and processes various data acquired by the biological information measurement apparatus 50. Specifically, the control unit 550 performs processing to detect a first feature, which is a characteristic feature of the electrocardiogram waveform, based on electrocardiogram information relating to the measured electrocardiogram waveform of the measurement subject. Further, the control unit 550 performs processing for detecting a second feature, which is a characteristic feature of the pulse wave, based on the pulse wave information relating to the measured pulse wave of the measurement subject. In addition, the control unit 550 performs processing for calculating the pulse wave transit time of the measurement subject based on the acquired electrocardiogram information and the pulse wave information. In the following, the configuration of the control unit 550 will be described in more detail.

The control unit 550 includes a biological information acquisition unit 551, a first feature detection unit 152, a second feature detection unit 242, a pulse wave measurement control unit 445, a power control unit 554, and a pulse wave transit time calculation unit 155. It is noted that, among the functions and structures of the control unit 550, those of the first feature detection unit 152, the second feature detection unit 242, the pulse wave measurement control unit 445, and the pulse wave transit time calculation unit 155 are the same as in the first and second embodiments of the present disclosure. Further, the functions and configuration of the power control unit 554 are the same as those of the power control unit 154 according to the first embodiment of the present disclosure and the power control unit 244 according to the second embodiment of the present disclosure.

The biological information acquisition unit 551 acquires biological information relating to the biological activity of the measurement subject. Here, the biological information may be any information about the biological activity of the measurement subject.

In the present embodiment, as biological information, the biological information acquisition unit 551 acquires first waveform information relating to a first waveform representing the measurement subject's beat that was measured at a first measurement site. Specifically, this first waveform information may be electrocardiogram information relating to the electrocardiogram waveform measured at the measurement subject's chest by the electrocardiogram measurement unit 110. Further, as biological information, the biological information acquisition unit 551 acquires second waveform information relating to a second waveform representing the measurement subject's pulse that was measured at a second measurement site. Specifically, this second waveform information may be pulse wave information relating to the pulse wave measured at a finger on the measurement subject's hand by the pulse wave measurement unit 210. The biological information acquisition unit 551 transmits the acquired electrocardiogram information to the first feature detection unit 152. Further, the biological information acquisition unit 551 transmits the acquired pulse wave information to the second feature detection unit 242.

The first feature detection unit 152 detects the first feature, which is a characteristic feature of the electrocardiogram waveform, based on the electrocardiogram information. In the present embodiment, the first feature detection unit 152 detects the initial rise point of the R wave in the electrocardiogram waveform as the first feature. The first feature detection unit 152 transmits information relating to the detected initial rise point of the R wave to the pulse wave measurement control unit 445 and the pulse wave transit time calculation unit 155.

The second feature detection unit 242 detects the second feature, which is a characteristic feature of the pulse wave, based on the pulse wave information. In the present embodiment, the second feature detection unit 242 detects the initial rise point of the pulse wave as the second feature. It is noted that the second feature detection unit 242 can detect the initial rise point by differentiating the pulse wave with respect to time twice, as described in “3.1. Pulse wave transit time calculation method”. The second feature detection unit 242 transmits information relating to the detected initial rise point to the pulse wave transit time calculation unit 155.

The pulse wave measurement control unit 445 controls the measurement of the measurement subject's pulse wave by controlling the drive of the pulse wave measurement unit 210. Specifically, the pulse wave measurement control unit 445 may control the measurement of the measurement subject's pulse wave by irradiating light from the light-emitting element 212 just for the pulse wave measurement period described with reference to FIG. 15. It is noted that to determine the pulse wave measurement period, the pulse wave measurement control unit 445 can utilize the transmitted information relating to the initial rise point of the R wave in the electrocardiogram waveform and information relating to the pulse wave transit time measured the last time.

The power control unit 554 controls the power unit 540 so that power is supplied to each of the constituent parts in the biological information measurement apparatus 50. For example, the power control unit 554 controls the power unit 540 so that power is supplied to the pulse wave measurement unit 210 during the above-described pulse wave measurement period.

The pulse wave transit time calculation unit 155 calculates the pulse wave transit time, which is the difference between the time corresponding to the first feature and the time corresponding to the second feature, based on the electrocardiogram information and the pulse wave information. In the example illustrated in FIG. 16, the pulse wave transit time calculation unit 155 calculates the pulse wave transit time by receiving the information relating to the first feature from the first feature detection unit 152 and the information relating to the second feature from the second feature detection unit 242. The information relating to the pulse wave transit time calculated by the pulse wave transit time calculation unit 155 is transmitted to an arbitrary external device via the communication unit 130, and the blood pressure value of the measurement subject is calculated by that external device based on the pulse wave transit time.

In the above, the configuration of the biological information measurement apparatus 50 according to the third embodiment of the present disclosure was described with reference to FIG. 16. Next, the various processing steps performed by the above-described biological information measurement apparatus 50 will be described with reference to a flow diagram.

### (5.3. Pulse Wave Transit Time Measurement Sequence)

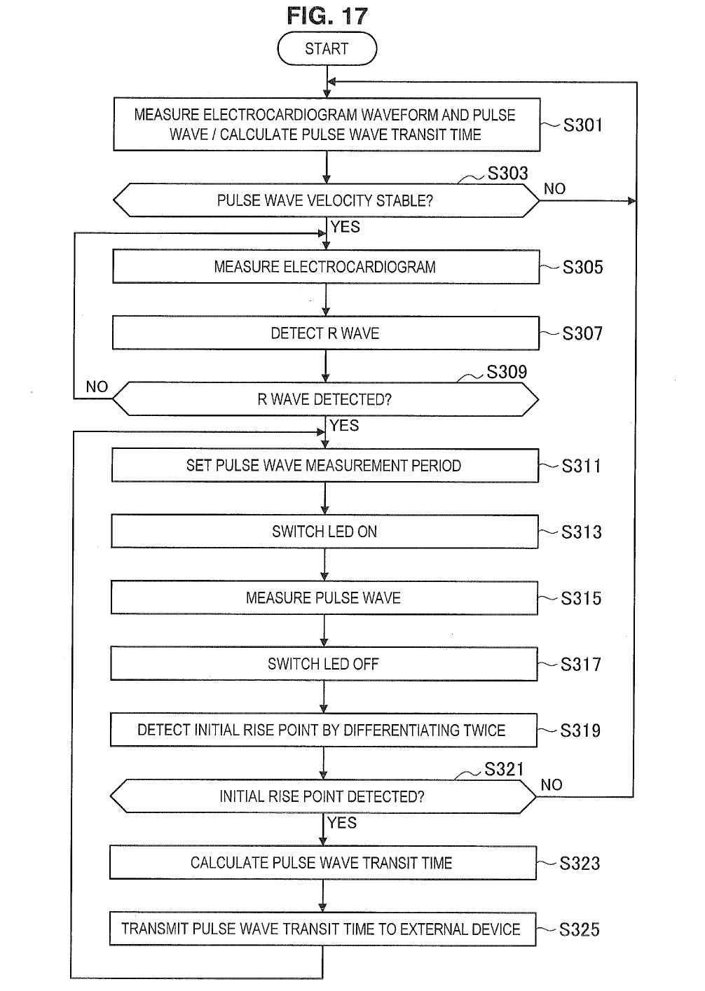

A pulse wave transit time calculation method according to the third embodiment of the present disclosure will now be described with reference to FIG. 17. FIG. 17 is a flow diagram illustrating a pulse wave transit time calculation method according to the third embodiment of the present disclosure.

First, in step S301, an electrocardiogram waveform and a pulse wave are measured, and based on those results, the pulse wave transit time is calculated. Here, in step S301, the electrocardiogram waveform and the pulse wave may be continuously measured without setting a pulse wave measurement period when measuring the electrocardiogram waveform and the pulse wave.

Next, it is determined whether the pulse wave transit time value calculated in step S301 is stable (step S303). It is noted that the determination regarding whether the pulse wave transit time is stable may be carried out based on whether a difference in the continuously-measured pulse wave transit time value is equal to or less than a predetermined threshold, for example. If it is determined that the pulse wave transit time value is not stable, the processing returns to step S301, the electrocardiogram waveform and pulse wave are measured again, and the pulse wave transit time is calculated. Namely, the processing of steps S301 and S303 is repeated until the pulse wave transit time can be stably acquired.

If it is determined that the pulse wave transit time value is stable, the processing proceeds to step S305. In step S305, electrocardiogram measurement of the measurement subject is carried out by the electrocardiogram measurement unit 110.

Next, an R wave is detected from the measured electrocardiogram waveform (step S307). Specifically, the initial rise point of an R wave in the electrocardiogram waveform is detected by the first feature detection unit 152.

Next, in step S309, it is determined whether an R wave was detected from the electrocardiogram waveform. If an R wave was not detected from the electrocardiogram waveform, the processing returns to step S305, and electrocardiogram measurement is carried out again.

If an R wave was detected from the electrocardiogram waveform, the processing proceeds to step S311. In step S311, a pulse wave measurement period is set based on the time corresponding to the detected initial rise point of the R wave and the stable pulse wave transit time calculated in step S301. It is noted that this pulse wave measurement period may be a period corresponding to the time width of the measurement window 750 illustrated in FIG. 15, for example.

Next, the light-emitting element 212, for example an LED, in the pulse wave measurement unit 210 is driven unit the control of the pulse wave measurement control unit 445 (step S331). Then, the measurement subject's pulse wave is measured (step S315), and after a predetermined duration has elapsed, the drive of the LED is stopped (step S317). It is noted that the duration from after the LED is driven in step S315 until the LED is stopped being driven in step S317 is a time corresponding to the pulse wave measurement period set in step S311.

Next, the initial rise point is detected from the measured pulse wave (step S319). Specifically, the second feature detection unit 242 detects the initial rise point by differentiating the pulse wave with respect to time twice.

Next, in step S321, it is determined whether an initial rise point was detected from the pulse wave. If an initial rise point was not detected from the pulse wave, the processing returns to step S311, and pulse wave measurement is carried out again.

If an initial rise point was detected from the pulse wave, the processing proceeds to step S323. In step S323, the pulse wave transit time, which is the difference between the time corresponding to the first feature and the time corresponding to the second feature, is calculated based on the electrocardiogram information and the pulse wave information (step S323). Specifically, the time corresponding to the first feature may be the time corresponding to the initial rise point of the R wave in the electrocardiogram waveform detected in step S307, and the time corresponding to the second feature may be the timing corresponding to the initial rise point of the pulse wave detected in step S319.

After the pulse wave transit time has been detected, in step S325, information relating to this pulse wave transit time is transmitted to an arbitrary external device. Then, the measurement subject's blood pressure is calculated by that external device based on the pulse wave transit time.

By repeating the above-described processing performed in steps S301 to S325, the pulse wave transit time and even the blood pressure of the measurement subject can be constantly continuously measured.

In the above, the third embodiment of the present disclosure was described with reference to FIGS. 15, 16, and 17. In the third embodiment of the present disclosure, the electrocardiogram measurement unit 110 measures the electrocardiogram waveform of the measurement subject. Further, the pulse wave measurement unit 210 starts up the pulse wave measurement unit for just the pulse wave measurement period, which is a predetermined duration, and measures the pulse wave of the measurement subject. Thus, by limiting the duration for measuring the pulse wave, the power consumed by the biological information measurement apparatus 50 can be decreased compared with when the pulse wave is constantly measured. Further, since the amount of information relating to the measured pulse wave is reduced, the amount of information handled during the series of processes for calculating the pulse wave transit time is reduced, which enables the power consumption of the biological information measurement apparatus 50 to be reduced decreased even further.

### 6. MODIFIED EXAMPLES

Next, modified examples according to the first, second, and third embodiments of the present disclosure will be described.

### (6.1. First Waveform)

In the above description, although cases were described in which the first waveform is an electrocardiogram waveform, the present disclosure is not limited to this. The first waveform may be some other waveform, as long as that waveform represents the measurement subject's beat. For example, the first waveform may be a waveform representing the heart sound of the measurement subject. Further, the first waveform may be a pulse wave measured at a measurement site different from the pulse wave serving as the second waveform.

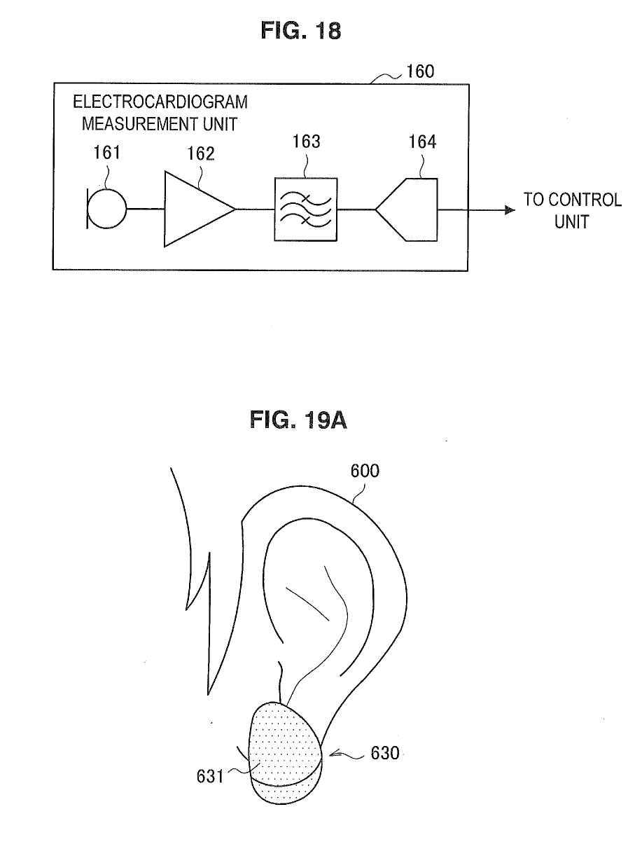

If the first waveform is the heart sound, instead of the electrocardiogram measurement unit 110 illustrated in FIGS. 8, 12, and 16, the first waveform is measured using a heart sound measurement unit. The configuration of such a heart sound measurement unit will be described with reference to FIG. 18. FIG. 18 is a schematic diagram illustrating a configuration example of a heart sound measurement unit when the first waveform is a heart sound. It is noted that since the processing performed after the heart sound that is measured with this heart sound measurement unit has been transmitted to the control unit is the same as the processing performed on the electrocardiogram waveform by the control units 150, 350, and 550 in the above “3. First embodiment of the present disclosure”, “4. Second embodiment of the present disclosure”, and “5. Third embodiment of the present disclosure”, a detailed description thereof will be omitted here.

As illustrated in FIG. 18, a heat sound measurement unit 160 according to the present modified example is configured from, for example, a microphone 161, a microphone amplifier 162, a bandpass filter 163, and an AD converter 164.

The microphone 161, which is, for example, a condenser microphone, inputs signal relating to heart sound to the microphone amplifier 162.

The microphone amplifier 162 amplifies the input signal relating to heart sound, and inputs the amplified signals to the bandpass filter 163. The bandpass filter 163 removes the frequency components other a desired band from the input signal relating to heart sound, and inputs the resultant signal to the AD converter 164. Here, the microphone amplifier 162 gain and the cutoff band and the like of the bandpass filter 163 can be appropriately set in consideration of the accuracy of the heart sound measurement value, the subsequent signal processing method and the like.

The AD converter 164 converts the analog signal relating to heart sound input from the bandpass filter 163 into a digital signal, and transmits the digital signal to the control units 150, 350, and 550.

It is noted that if the first waveform is a heart sound, the first feature, which is a characteristic feature of a waveform representing heart sound, may be a point representing an I sound, for example. Therefore, in the present modified example, the first feature detection unit 152 in the control units 150, 350, and 550 detects a point representing the I sound from the waveform representing heart sound as the first feature. Further, a time corresponding to the point representing the I sound is used as the time T1 corresponding to the first feature in the subsequent calculation of the pulse wave transit time.

In addition, if the first waveform is a heart sound, instead of the R wave detection packet 720, an I sound detection packet may be used for the electrocardiogram waveform transmitted from the electrocardiogram information measurement apparatuses 10 and 30 to the pulse wave information measurement apparatuses 20 and 40. Further, the reception window 730 of the HBC reception unit 120 and the HBC transmission and reception unit 320 may be set based on the time at which the pulse wave information measurement apparatuses 20 and 40 detected the I sound detection packet. In addition, the measurement window 750 of the pulse wave measurement unit 210 may be set based on the time at which the pulse wave information measurement apparatuses 20 and 40 received the I sound detection packet. It is noted that the configuration of the I sound detection packet may be the same as the configuration of the pulse wave detection packet 710 and the R wave detection packet 720.

Further, if the first waveform is a waveform measured at a measurement site different from the second waveform, instead of the electrocardiogram measurement unit 110 illustrated in FIGS. 8, 12, and 16, the first waveform is measured using the pulse wave measurement unit 210 illustrated in FIGS. 9, 13, and 16. In addition, in such a case, the same processing as described in the above “3. First embodiment of the present disclosure”, “4. Second embodiment of the present disclosure”, and “5.

Third embodiment of the present disclosure” is carried out for the processing of the initial rise point of the pulse wave acting as the first waveform as the first feature.

### (6.2. Pulse Wave Measurement Site)

Although cases were described above in which the measurement site for measuring the pulse wave (the second measurement site) is a finger on the measurement subject's hand, the present disclosure is not limited to this example. The second measurement site may be a site other than on a finger on the hand, such as an ear for example.

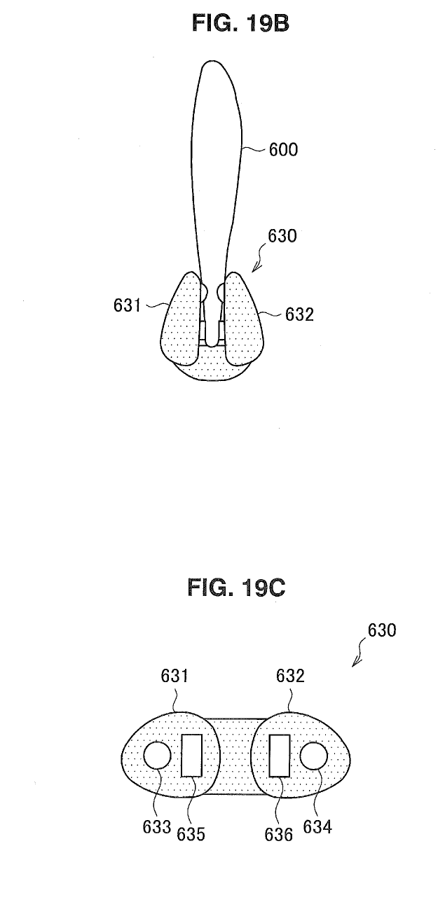

The configuration of the pulse wave information measurement apparatus when the second measurement site is the measurement subject's ear will be described with reference to FIGS. 19A to 19C. FIGS. 19A to 19C are schematic diagrams illustrating appearance examples of a pulse wave information measurement apparatus when the second measurement site is the measurement subject's ear. It is noted that since the function and the configuration of this pulse wave information measurement apparatus are the same as the function and configuration of the pulse wave information measurement apparatuses 20 and 40 in the above “3. First embodiment of the present disclosure” and “4. Second embodiment of the present disclosure”, a detailed description thereof will be omitted here.

It is noted that in the following description of the present modified example, a modified example of the first embodiment of the present disclosure and of the second embodiment of the present disclosure will be described. However, the present modified example can also be applied to the third embodiment of the present disclosure. When applying the present modified example to the third embodiment of the present disclosure, FIGS. 19A to 19C correspond to an illustration of a modified example of the pulse wave measurement unit 210 of the biological information measurement apparatus 50. In such a case, since the internal function and configuration in the modified example of the pulse wave measurement unit 210 illustrated in FIGS. 19A to 19C are the same as the function and configuration of the pulse wave measurement unit 210 in the above “5. Third embodiment of the present disclosure”, a detailed description thereof will be omitted here.

As illustrated in FIGS. 19A to 19C, a pulse wave information measurement apparatus 630 according to the present modified example has a clip shape, and is worn so as to sandwich a partial area of an ear, which is one site on a body 600, between a first measurement unit 631 and a second measurement unit 632. FIG. 19A is a front view illustrating the pulse wave information measurement apparatus 630 worn on an ear. FIG. 19B is a side view illustrating the pulse wave information measurement apparatus 630 worn on an ear.

Further, FIG. 19C is a schematic diagram illustrating the pulse wave information measurement apparatus 630 in an open state. As illustrated in FIG. 19C, a light-emitting element 634 and an electrode 635 are provided on the face in contact with the ear that is the measurement site of the first measurement unit 631. The light-emitting element 634 is, for example, a light-emitting diode (LED) that irradiates infrared light. Further, a light-emitting element 636 and an electrode 637 are provided on the face in contact with the ear that is the measurement site of the second measurement unit 632. The light-receiving element 636 is, for example, a photodiode. Further, as illustrated in FIG. 19B, for example, the light-emitting element 634 and the light-emitting element 636 are provided at a position sandwiching the ear while facing each other when the pulse wave information measurement apparatus 630 is worn on the ear.

Here, the light-emitting element 634 and the light-emitting element 636 correspond to the light-emitting element 623 and the light-receiving element 624 illustrated in FIG. 5B and to the light-emitting element 212 and the light-receiving element 213 illustrated in FIGS. 9 and 13. Namely, the pulse wave information measurement apparatus 630 according to the present modified example can measure the pulse wave of the measurement subject by detecting light irradiated from the light-emitting element 634 that has passed through and/or been scattered by the ear.

Further, the electrodes 635 and 637 correspond to the electrodes 626 and 627 illustrated in FIG. 5B and to the electrodes 221a and 221b illustrated in FIGS. 9 and 13. Namely, the pulse wave information measurement apparatus 630 according to the present modified example can exchange various types of information with an electrocardiogram information measurement apparatus based on human body communication by using the electrodes 635 and 637 as electrodes for human body communication.

### (6.3. Pulse Wave Transit Time Calculation Unit)

In the above “4. Second embodiment of the present disclosure”, although a case was described in which the electrocardiogram information measurement apparatus 30 has a function for calculating the pulse wave transit time, the present disclosure is not limited to this. In a case in which the electrocardiogram information measurement apparatus 30 and the pulse wave information measurement apparatus 40 are exchanging various types of information with each other like in the second embodiment of the present disclosure, the pulse wave transit time may be calculated by the pulse wave information measurement apparatus 40.

If the pulse wave information measurement apparatus 40 has a function for calculating the pulse wave transit time, the pulse wave transit time is calculated in the following order, for example.

First, an electrocardiogram waveform of the measurement subject is measured with the electrocardiogram information measurement apparatus. The electrocardiogram information measurement apparatus detects a first feature (e.g., an initial rise point of an R wave) from the measured electrocardiogram waveform, and transmits an R wave detection packet 720 and information relating to the pulse wave transit time measured the last time as electrocardiogram information to the pulse wave information measurement apparatus.

At the pulse wave information measurement apparatus, a pulse wave measurement period, which is a predetermined duration, is set based on the transmitted R wave detection packet 720 and information relating to the pulse wave transit time, and the measurement subject's pulse wave is measured for that pulse wave measurement period. Further, a second feature (e.g., an initial rise point of the pulse wave) is detected. Then, based on the R wave detection packet transmitted from the electrocardiogram information measurement apparatus and the detected second feature, the pulse wave transit time, which is the difference between the timing corresponding to the first feature and the timing corresponding to the second feature, is calculated.

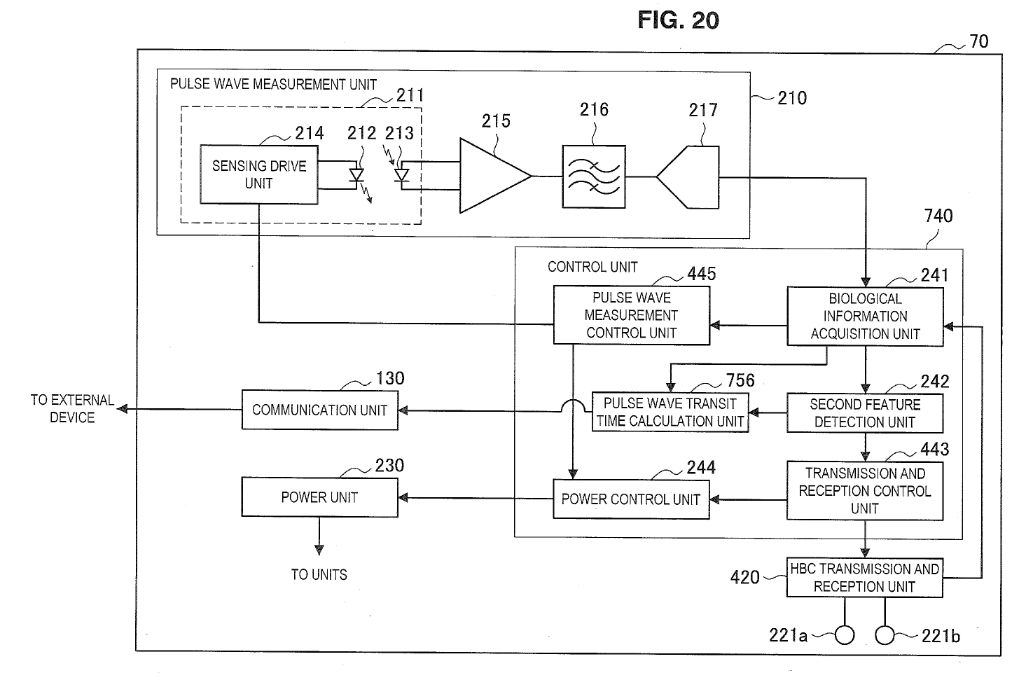

The configuration of a pulse wave information measurement apparatus when the pulse wave information measurement apparatus 40 has the function for calculating the pulse wave transit time will now be described with reference to FIG. 20. FIG. 20 is a function block diagram illustrating the configuration of a pulse wave information measurement apparatus when the pulse wave information measurement apparatus 40 has a function for calculating a pulse wave transit time.

As illustrated in FIG. 20, a pulse wave information measurement apparatus 70 according to the present modified example includes a pulse wave measurement unit 210, an HBC transmission and reception unit 420, a power unit 230, a control unit 740, and a communication unit 130. It is noted that the pulse wave information measurement apparatus 70 corresponds to the pulse wave information measurement apparatus 40 according to the second embodiment of the present disclosure, to which the communication unit 130 has been added, and a below-described pulse wave transit time calculation unit 756 has been further added to the control unit 740. Further, the function and configuration of the communication unit 130 are the same as the function and configuration of the communication unit 130 in the electrocardiogram information measurement apparatuses 10 and 30 and the biological information measurement apparatus 50 illustrated in FIGS. 8, 12, and 16. Therefore, the following description of the pulse wave information measurement apparatus 70 will mainly be about the function and configuration of the control unit 740, and a detailed description of the other structures will be omitted here.

The control unit 740 controls the pulse wave information measurement apparatus 70 in an integrated manner, and processes various data acquired by the pulse wave information measurement apparatus 70. Specifically, the control unit 740 performs processing to detect a second feature, which is a characteristic feature of a pulse wave, based on pulse wave information relating to the measured pulse wave of the measurement subject. Further, the control unit 740 controls the HBC transmission and reception unit 740, and performs processing for receiving the electrocardiogram information (R wave detection packet 720) and information relating to the pulse wave transit time transmitted from the electrocardiogram information measurement apparatus. In addition, the control unit 740 performs processing to calculate the pulse wave transit time of the measurement subject based on the pulse wave information and the received electrocardiogram information. In the following, the configuration of the control unit 740 will be described in more detail.

The control unit 740 has a biological information acquisition unit 241, a second feature detection unit 242, a power control unit 244, a transmission and reception control unit 443, a pulse wave measurement control unit 445, and the pulse wave transit time calculation unit 756. Here, since the function and configuration of the biological information acquisition unit 241, the second feature detection unit 242, the power control unit 244, the transmission and reception control unit 443, and the pulse wave measurement control unit 445 are the same as the biological information acquisition unit 241, the second feature detection unit 242, the transmission and reception control unit 443, and the pulse wave measurement control unit 445 in the pulse wave information measurement apparatuses 20 and 40 illustrated in FIGS. 9 and 13, a detailed description thereof will be omitted here.

The pulse wave transit time calculation unit 756 calculates the pulse wave transit time, which is the difference between the timing corresponding to the first feature and the timing corresponding to the second feature, based on the electrocardiogram information and the pulse wave information. In the present modified example, the pulse wave transit time calculation unit 756 calculates the pulse wave transit time by receiving information relating to the first feature from the electrocardiogram information measurement apparatus via the HBC transmission and reception unit 420 and the biological information acquisition unit 241, and receiving the information relating to the second feature from the second feature detection unit 242. The information relating to the pulse wave transit time calculated by the pulse wave transit time calculation unit 756 is transmitted to an arbitrary external device via the communication unit 130, and the blood pressure value of the measurement subject is calculated by that external device based on the pulse wave transit time.

### 7. HARDWARE CONFIGURATION

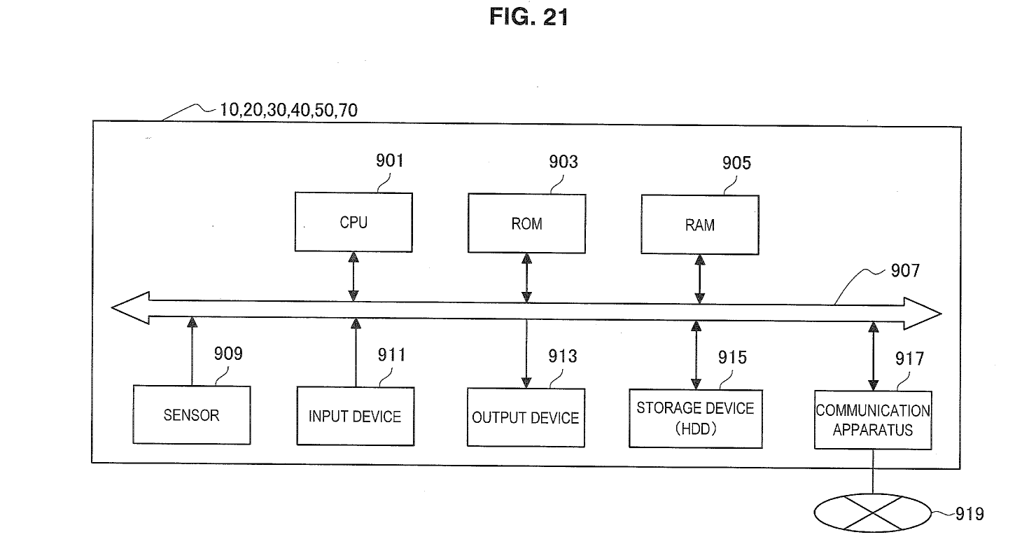

Next, the hardware configuration of the biological information measurement apparatuses according to the first, second, and third embodiments of the present disclosure will be described with reference to FIG. 21. FIG. 21 is a function block diagram illustrating an example of the hardware configuration of the biological information measurement apparatuses according to the first, second, and third embodiments of the present disclosure.

The biological information measurement apparatuses 10, 20, 30, 40, 50, and 70 mainly include a CPU 901, a ROM 903, and a ROM 905. Further, the biological information measurement apparatuses 10, 20, 30, 40, 50, and 70 further include an internal bus 907, a sensor 909, an input device 911, an output device 913, a storage device 915, and a communication apparatus 917.

The CPU 901, which functions as a calculation processing apparatus and a control apparatus, controls all or a part of the operations in the biological information measurement apparatuses 10, 20, 30, 40, 50, and 70 based on various programs recorded in the ROM 903, RAM 905, or storage device 915. The CPU 901 corresponds to, for example, in the respective embodiments of the present disclosure, the control units 150, 240, 350, 440, 550, and 740. The ROM 903 stores programs, calculation parameters and the like used by the CPU 901. The RAM 905 temporarily stores the programs to be used by the CPU 901, and parameters that are appropriately changed during program execution. The CPU 901, ROM 903, and ROM 905 are, for example, connected to each other by the internal bus 907, which is configured from a bus such as a CPU bus. Further, various interfaces, namely, the sensor 909, the input device 911, the output device 913, the storage device 915, and the communication apparatus 917, are connected to the internal bus 907.

The sensor 909 is a detection unit for, for example, detecting biological information unique to a user or detecting various pieces of information used for acquiring such biological information. In the respective embodiments of the present disclosure, the sensor 909 corresponds to the electrocardiogram measurement unit 110, the pulse wave measurement unit 210, and the heart sound measurement unit 160. Further, as another example different from these, the sensor 909 may have various types of image sensor, such as a CCD (charge-coupled device) or a CMOS (complementary metal oxide semiconductor). If the sensor 909 does have various types of image sensor, the sensor 909 may further have an optical system such as a lens, a light source and the like, that are used for capturing an image of a biological site. In addition to the above-described parts, the sensor 909 may also include various known measurement devices, such as a thermometer, an illuminance meter, a hygrometer, a speedometer, an accelerometer and the like.

Here, although not clearly illustrated in FIG. 8, 9, 12, 13, or 16, the biological information measurement apparatuses 10, 20, 30, 40, 50, and 70 may further include the input device 911, the output device 913, and the storage device 915.

The input device 911 is, for example, a touch panel, button, switch or the like that is operated by the user. Further, the input device 911 may be, for example, a remote control device (a so-called “remote control”) that utilizes infrared rays or other radio waves. Moreover, the input device 911 is configured from, for example, an input control circuit that generates an input signal based on information input by the user using the above-described operation device, and outputs the generated input signal to the CPU 901. The user of the biological information measurement apparatuses 10, 20, 30, 40, 50, and 70 can input various types of data and issue processing operation instructions to the biological information measurement apparatuses 10, 20, 30, 40, 50, and 70 by operating this input device 911.

The output device 913 is configured from a device that can visually or aurally notify the user of acquired information. Examples of such a device include a display device such as a CRT display device, a liquid crystal display device, a plasma display panel device, an EL display device, and a lamp, an audio output device such as a speaker or headphones and the like. The output device 913 outputs results obtained based on various processes performed by the biological information measurement apparatuses 10, 20, 30, 40, 50, and 70, for example. Specifically, the display apparatus displays results obtained based on various processes performed by the biological information measurement apparatuses 10, 20, 30, 40, 50, and 70 as text or an image. In the respective embodiments of the present disclosure, the display apparatus can display, for example, information relating to the measured electrocardiogram waveform and information relating to the measured pulse wave of the measurement subject. Further, the audio device may output an alarm sound, a buzzer or the like via a speaker in order to send a message to the user that the series of measurements relating to pulse wave transit time calculation has finished, for example.

The storage device 915 is a device for storing data that is configured as an example of the storage unit in the biological information measurement apparatuses 10, 20, 30, 40, 50, and 70. The storage device 915 is configured from, for example, a magnetic storage unit device such as a HDD (hard disk drive), a semiconductor storage device, an optical storage device, a magneto-optical storage device and the like. The storage device 915 stores programs and various types of data executed by the CPU 901, and various types of externally-acquired data. For example, in the respective embodiments of the present disclosure, the storage device 915 can store information relating to the measured electrocardiogram waveform and information relating to the measured pulse wave of the measurement subject, as well as information relating to the calculated pulse wave transit time.

The communication apparatus 917 is a communication interface configured from a communication device for connecting to a communication network 919, for example. In the respective embodiments of the present disclosure, the communication apparatus 917 corresponds to the communication unit 130. The communication apparatus 917 may be a wired or a wireless LAN (local area network), Bluetooth®, or WUSB (wireless USB) communication card, for example. Further, the communication apparatus 917 may be an optical communication router, an ADSL (asymmetric digital subscriber line) router, or a modem used for various types of communication. This communication apparatus 917 can, for example, transmit and receive signals and the like based on a predetermined protocol such as TCP/IP, for example, to/from the Internet or another communication device. In addition, the communication network 919 connected to the communication apparatus 917 is configured from a wired or wirelessly connected network, and may be, for example, the Internet, a home LAN, infrared communication, radio wave communication, satellite communication or the like.

Further, although not clearly illustrated in FIG. 21, the biological information measurement apparatuses 10, 20, 30, 40, 50, and 70 may further include a drive for performing a write operation and a read operation of the information into/from various types of removable storage media. In addition, the biological information measurement apparatuses 10, 20, 30, 40, 50, and 70 can also further include a connection port that is directly connected to various external devices for transmitting information to/from those external devices. If the biological information measurement apparatuses 10, 20, 30, 40, 50, and 70 include a drive and a connection port, the various types of information transmitted via the communication apparatus 917 can be transmitted by this drive and/or connection port. However, an appropriate hardware configuration of the biological information measurement apparatuses 10, 20, 30, 40, 50, and 70 may be selected in consideration of reducing the power consumption of the apparatus.

In the above, an example of a hardware configuration capable of realizing the functions of the biological information measurement apparatuses 10, 20, 30, 40, 50, and 70 according to the embodiments of the present disclosure was described. The above-described constituent parts may be configured using multi-purpose members or from hardware specialized for the function of each constituent part.

Therefore, the utilized hardware configuration may be appropriately modified based on the technological level at the time of implementing the embodiments of the present disclosure.

Note that there may be produced a computer program for realizing each function of the biological information measurement apparatuses 10, 20, 30, 40, 50, and 70 according to the above-described embodiments of the present disclosure, and the computer program can be implemented in a personal computer or the like. Further, there can also be provided a computer-readable recording medium having the computer program stored therein. Examples of the recording medium include a magnetic disk, an optical disc, a magneto-optical disk, a flash memory and the like. Further, the computer program may be distributed via a network, without using a recording medium, for example.

### 8. CONCLUSION

As described above, the biological information measurement apparatus, biological information measurement system, and biological information measurement method according to the first, second, and third embodiments of the present disclosure can obtain the following advantageous effects.

In the first embodiment of the present disclosure, the electrocardiogram information measurement apparatus 10 measures the electrocardiogram waveform of the measurement subject, and the pulse wave information measurement apparatus 20 measures the pulse wave of the measurement subject. Further, the pulse wave detection packet 710 is transmitted from the pulse wave information measurement apparatus 20 to the electrocardiogram information measurement apparatus 10 as information relating to the pulse wave, and the pulse wave transit time is calculated based on electrocardiogram information relating to the electrocardiogram waveform and the pulse wave information. Thus, by transmitting and receiving only information relating to the timing corresponding to the second feature, which is a characteristic feature of the pulse wave, as pulse wave information rather than all the information relating to the pulse wave (the waveform data itself), the amount of data that is handled can be reduced, and a decrease in power consumption of the electrocardiogram information measurement apparatus 10 and the pulse wave information measurement apparatus 20 can be realized. Further, by reducing power consumption, the battery mounted in the electrocardiogram information measurement apparatus 10 and the pulse wave information measurement apparatus 20 can be made more compact, so that even better portability is achieved, and user friendliness for the measurement subject is improved.

Further, in the first embodiment of the present disclosure, the HBC reception unit 120 in the electrocardiogram information measurement apparatus 10 and the HBC transmission unit 220 in the pulse wave information measurement apparatus 20 are started up at a timing when data is transmitted from the pulse wave information measurement apparatus 20 to the electrocardiogram information measurement apparatus 10. Thus, by limiting the running time of the HBC reception unit 120 and the HBC transmission unit 220, the power consumption of the electrocardiogram information measurement apparatus 10 and the pulse wave information measurement apparatus 20 can be reduced even further.

In addition, in the first embodiment of the present disclosure, the transmission of data between the electrocardiogram information measurement apparatus 10 and the pulse wave information measurement apparatus 20 was performed using human body communication. Thus, by using human body communication that has a lower power consumption than other forms of wireless communication as the communication method between the two apparatuses, even further reductions in the power consumption of the electrocardiogram information measurement apparatus 10 and the pulse wave information measurement apparatus 20 are realized.

Further, in the first embodiment of the present disclosure, by thus using human body communication, a cable or other such connection between the electrocardiogram information measurement apparatus 10 and the pulse wave information measurement apparatus 20 does not have to be used. For example, when sleeping while wearing the electrocardiogram information measurement apparatus 10 and the pulse wave information measurement apparatus 20 in order to measure blood pressure while asleep, the presence of a cable or other such connection can hinder stable blood pressure measurement due to contact or interference with other objects caused by unintentional movements such as turning in bed. In the first embodiment of the present disclosure, since the use of human body communication makes it unnecessary to use a cable or other such connection, more stable blood pressure measurement is realized and user friendliness for the measurement subject is improved.

In addition, in the first embodiment of the present disclosure, in the electrocardiogram information measurement apparatus 10, the electrodes for human body communication are also used as the electrodes for electrocardiogram measurement. Therefore, the number of added structures for human body communication can be comparatively less, so that the electrocardiogram information measurement apparatus 10 can be more compact and have better portability.

Moreover, in addition to the advantageous effects of the above-described first embodiment of the present disclosure, in the second embodiment of the present disclosure the following advantageous effects can be obtained.

In the second embodiment of the present disclosure, the electrocardiogram information measurement apparatus 30 measures the electrocardiogram waveform of the measurement subject, and the pulse wave information measurement apparatus 40 starts up the pulse wave measurement unit for just the pulse wave measurement period, which is a predetermined duration, and measures the pulse wave of the measurement subject. Thus, by limiting the duration for measuring the pulse wave, the power consumed by the electrocardiogram information measurement apparatus 30 and the pulse wave information measurement apparatus 40 can be decreased compared with when the pulse wave is constantly measured.

Further, in the second embodiment of the present disclosure, the R wave detection packet 720 and the pulse wave transit time measured the last time are transmitted from the electrocardiogram information measurement apparatus 30 to the pulse wave information measurement apparatus 40, and the pulse wave detection packet 710 is transmitted from the pulse wave information measurement apparatus 40 to the electrocardiogram information measurement apparatus 30. Thus, by transmitting and receiving only information relating to the timing corresponding to the time corresponding to the first feature and the second feature, which are characteristic features of the electrocardiogram waveform and the pulse wave, as electrocardiogram information and the pulse wave information rather than all the information relating to the electrocardiogram waveform and the pulse wave (the waveform data itself), the amount of data that is handled can be reduced, and a decrease in power consumption of the electrocardiogram information measurement apparatus 30 and the pulse wave information measurement apparatus 40 can be realized.

Further, in the second embodiment of the present disclosure, the HBC transmission and reception unit 320 in the electrocardiogram information measurement apparatus 30 and the HBC transmission and reception unit 420 in the pulse wave information measurement apparatus 40 are started up at a timing when data is exchanged between the electrocardiogram information measurement apparatus 30 and the pulse wave information measurement apparatus 40. Thus, by limiting the running time of the HBC transmission and reception unit 320 and the HBC transmission and reception unit 420, the power consumption of the electrocardiogram information measurement apparatus 10 and the pulse wave information measurement apparatus 20 can be reduced even further.

Still further, in the third embodiment of the present disclosure, the electrocardiogram measurement unit 110 measures the electrocardiogram waveform of the measurement subject. Further, the pulse wave measurement unit 210 starts up the pulse wave measurement unit for just the pulse wave measurement period, which is a predetermined duration, and measures the pulse wave of the measurement subject. Thus, by limiting the duration for measuring the pulse wave, the power consumed by the biological information measurement apparatus 50 can be decreased compared with when the pulse wave is constantly measured. Further, since the amount of information relating to the measured pulse wave is reduced, the amount of information handled during the series of processes for calculating the pulse wave transit time is reduced, which enables the power consumption of the biological information measurement apparatus 50 to be reduced decreased even further.

It should be understood by those skilled in the art that various modifications, combinations, sub-combinations and alterations may occur depending on design requirements and other factors insofar as they are within the scope of the appended claims or the equivalents thereof.

Additionally, the present technology may also be configured as below.

(1) A biological information measurement apparatus including: a biological information acquisition unit configured to acquire at least first waveform information relating to a first waveform representing a beat of a measurement subject measured at a first measurement site, and as second waveform information relating to a second waveform representing the beat of the measurement subject measured at a second measurement site different from the first measurement site, information relating to a timing corresponding to a second feature which is a characteristic feature of the second waveform; and

a pulse wave transit time calculation unit configured to, based on the first waveform information and the second waveform information, calculate a pulse wave transit time, which is a difference between a timing corresponding to a first feature which is a characteristic feature of the first waveform and the timing corresponding to the second feature which is a characteristic feature of the second waveform.

(2) The biological information measurement apparatus according to (1), further including:

a biological information reception unit configured to receive at least the second waveform information,

wherein the biological information reception unit is configured to be started up for just a second waveform information reception period which is a predetermined duration, and to receive the second waveform information during the second waveform information reception period.

(3) The biological information measurement apparatus according to (2), wherein the second waveform information reception period is determined based on a timing corresponding to the first feature of the first waveform.
(4) The biological information measurement apparatus according to any one of (1) to
(3), wherein the second waveform is measured for just a second waveform measurement period which includes a timing corresponding to the second feature and is shorter than a period of a beat of the second waveform.
(5) The biological information measurement apparatus according to (4), wherein the second waveform measurement period is determined based on a timing corresponding to the first feature of the first waveform and the pulse wave transit time.
(6) The biological information measurement apparatus according to any one of (1) to (5),

wherein at least one of the first waveform information and the second waveform information is transmitted by human body communication, and

wherein the biological information acquisition unit is configured to acquire the at least one of the first waveform information and the second waveform information via human body communication.

(7) The biological information measurement apparatus according to (2), wherein the biological information reception unit is configured to receive the second waveform information by human body communication from another apparatus.
(8) The biological information measurement apparatus according to (7), wherein the first waveform is an electrocardiogram waveform of the measurement subject,

wherein the second waveform is a pulse wave of the measurement subject,

wherein the biological information measurement apparatus further includes at least a pair of electrodes and an electrocardiogram measurement unit configured to measure the electrocardiogram waveform with the electrodes, and

wherein the biological information reception unit is configured to perform the human body communication via the electrodes.

(9) The biological information measurement apparatus according to (8), wherein the electrodes are connected to the biological information reception unit when the human body communication is performed.
(10) The biological information measurement apparatus according to (1), further including:

a first waveform measurement unit configured to measure the first waveform; and

a second waveform measurement unit configured to measure the second waveform,

wherein the second waveform measurement unit is configured to measure the second waveform for just a second waveform measurement period, which includes a timing corresponding to the second feature and is shorter than a period of the second waveform.

(11) The biological information measurement apparatus according to any one of (1) to (10),

wherein the first waveform is an electrocardiogram waveform of the measurement subject,

wherein the second waveform is a pulse wave of the measurement subject,

wherein the first feature of the first waveform is an initial rise point of an R wave in the electrocardiogram waveform, and

wherein the second feature of the second waveform is an initial rise point of the pulse wave.

(12) The biological information measurement apparatus according to any one of (1) to (10),

wherein the first waveform is a waveform representing a heart sound of the measurement subject,

wherein the second waveform is a pulse wave of the measurement subject, and

wherein the first feature of the first waveform is determined based on an I sound of the heart sound.

(13) The biological information measurement apparatus according to any one of (1) to (10), wherein the first waveform and the second waveform are measured at different measurement sites of the measurement subject.
(14) A biological information measurement system including:

a first waveform information measurement apparatus that includes a first waveform measurement unit configured to measure a first waveform representing a beat of a measurement subject at a first measurement site, and a first feature detection unit configured to detect a first feature which is a characteristic feature of the first waveform; and

a second waveform information measurement apparatus that includes a second waveform measurement unit configured to measure a second waveform representing the beat of the measurement subject at a second measurement site that is different from the first measurement site, a second feature detection unit configured to detect a second feature which is a characteristic feature of the second waveform, and a biological information transmission unit configured to transmit second waveform information relating to the measured second waveform,

wherein the first waveform information measurement apparatus further includes a biological information reception unit configured to receive the second waveform information, and a pulse wave transit time calculation unit configured to calculate a pulse wave transit time, which is a difference between a timing corresponding to the first feature and the timing corresponding to the second feature, and

wherein the biological information transmission unit is configured to transmit information relating to a timing corresponding to the second feature as the second waveform information.

(15) The biological information measurement system according to (14), wherein the second waveform measurement unit is configured to measure the second waveform for just a second waveform measurement period which includes a timing corresponding to the second feature and is shorter than a period of a beat of the second waveform.
(16) A biological information measurement method including:

acquiring at least first waveform information relating to a first waveform representing a beat of a measurement subject measured at a first measurement site, and as second waveform information relating to a second waveform representing the beat of the measurement subject measured at a second measurement site different from the first measurement site, information relating to a timing corresponding to a second feature which is a characteristic feature of the second waveform; and

calculating, based on the first waveform information and the second waveform information, a pulse wave transit time, which is a difference between a timing corresponding to a first feature which is a characteristic feature of the first waveform and the timing corresponding to the second feature which is a characteristic feature of the second waveform.

(17) A program for causing a computer to realize:

a function for acquiring at least first waveform information relating to a first waveform representing a beat of a measurement subject measured at a first measurement site, and as second waveform information relating to a second waveform representing the beat of the measurement subject measured at a second measurement site different from the first measurement site, information relating to a timing corresponding to a second feature which is a characteristic feature of the second waveform; and

a function for calculating, based on the first waveform information and the second waveform information, a pulse wave transit time, which is a difference between a timing corresponding to a first feature which is a characteristic feature of the first waveform and the timing corresponding to the second feature which is a characteristic feature of the second waveform.

What is claimed is:

1. A biological information measurement apparatus comprising:
a biological information acquisition unit configured to acquire at least first waveform information relating to a first waveform representing a beat of a measurement subject measured at a first measurement site, and as second waveform information relating to a second waveform representing the beat of the measurement subject measured at a second measurement site different from the first measurement site, information relating to a timing corresponding to a second feature which is a characteristic feature of the second waveform; and
a pulse wave transit time calculation unit configured to, based on the first waveform information and the second waveform information, calculate a pulse wave transit time, which is a difference between a timing corresponding to a first feature which is a characteristic feature of the first waveform and the timing corresponding to the second feature which is a characteristic feature of the second waveform.

2. The biological information measurement apparatus according to claim 1, further comprising:
a biological information reception unit configured to receive at least the second waveform information,
wherein the biological information reception unit is configured to be started up for just a second waveform information reception period which is a predetermined duration, and to receive the second waveform information during the second waveform information reception period.

3. The biological information measurement apparatus according to claim 2, wherein the second waveform information reception period is determined based on a timing corresponding to the first feature of the first waveform.

4. The biological information measurement apparatus according to claim 1, wherein the second waveform is measured for just a second waveform measurement period which includes a timing corresponding to the second feature and is shorter than a period of a beat of the second waveform.

5. The biological information measurement apparatus according to claim 4, wherein the second waveform measurement period is determined based on a timing corresponding to the first feature of the first waveform and the pulse wave transit time.

6. The biological information measurement apparatus according to claim 1,
wherein at least one of the first waveform information and the second waveform information is transmitted by human body communication, and
wherein the biological information acquisition unit is configured to acquire the at least one of the first waveform information and the second waveform information via human body communication.

7. The biological information measurement apparatus according to claim 2, wherein the biological information reception unit is configured to receive the second waveform information by human body communication from another apparatus.

8. The biological information measurement apparatus according to claim 7,
wherein the first waveform is an electrocardiogram waveform of the measurement subject,
wherein the second waveform is a pulse wave of the measurement subject,
wherein the biological information measurement apparatus further comprises at least a pair of electrodes and an electrocardiogram measurement unit configured to measure the electrocardiogram waveform with the electrodes, and
wherein the biological information reception unit is configured to perform the human body communication via the electrodes.

9. The biological information measurement apparatus according to claim 8, wherein the electrodes are connected to the biological information reception unit when the human body communication is performed.

10. The biological information measurement apparatus according to claim 1, further comprising:
a first waveform measurement unit configured to measure the first waveform; and
a second waveform measurement unit configured to measure the second waveform,
wherein the second waveform measurement unit is configured to measure the second waveform for just a second waveform measurement period, which includes a timing corresponding to the second feature and is shorter than a period of the second waveform.

11. The biological information measurement apparatus according to claim 1,
wherein the first waveform is an electrocardiogram waveform of the measurement subject,
wherein the second waveform is a pulse wave of the measurement subject,
wherein the first feature of the first waveform is an initial rise point of an R wave in the electrocardiogram waveform, and
wherein the second feature of the second waveform is an initial rise point of the pulse wave.

12. The biological information measurement apparatus according to claim 1,
wherein the first waveform is a waveform representing a heart sound of the measurement subject,
wherein the second waveform is a pulse wave of the measurement subject, and
wherein the first feature of the first waveform is determined based on an I sound of the heart sound.

13. The biological information measurement apparatus according to claim 1, wherein the first waveform and the second waveform are measured at different measurement sites of the measurement subject.

14. A biological information measurement system comprising:
a first waveform information measurement apparatus that includes a first waveform measurement unit configured to measure a first waveform representing a beat of a measurement subject at a first measurement site, and a first feature detection unit configured to detect a first feature which is a characteristic feature of the first waveform; and
a second waveform information measurement apparatus that includes a second waveform measurement unit configured to measure a second waveform representing the beat of the measurement subject at a second measurement site that is different from the first measurement site, a second feature detection unit configured to detect a second feature which is a characteristic feature of the second waveform, and a biological information transmission unit configured to transmit second waveform information relating to the measured second waveform,
wherein the first waveform information measurement apparatus further includes a biological information reception unit configured to receive the second waveform information, and a pulse wave transit time calculation unit configured to calculate a pulse wave transit time, which is a difference between a timing corresponding to the first feature and the timing corresponding to the second feature, and
wherein the biological information transmission unit is configured to transmit information relating to a timing corresponding to the second feature as the second waveform information.

15. The biological information measurement system according to claim 14, wherein the second waveform measurement unit is configured to measure the second waveform for just a second waveform measurement period which includes a timing corresponding to the second feature and is shorter than a period of a beat of the second waveform.

16. A biological information measurement method comprising:
acquiring at least first waveform information relating to a first waveform representing a beat of a measurement subject measured at a first measurement site, and as second waveform information relating to a second waveform representing the beat of the measurement subject measured at a second measurement site different from the first measurement site, information relating to a timing corresponding to a second feature which is a characteristic feature of the second waveform; and
calculating, based on the first waveform information and the second waveform information, a pulse wave transit time, which is a difference between a timing corresponding to a first feature which is a characteristic feature of the first waveform and the timing corresponding to the second feature which is a characteristic feature of the second waveform.

17. A program for causing a computer to realize:
a function for acquiring at least first waveform information relating to a first waveform representing a beat of a measurement subject measured at a first measurement site, and as second waveform information relating to a second waveform representing the beat of the measurement subject measured at a second measurement site different from the first measurement site, information relating to a timing corresponding to a second feature which is a characteristic feature of the second waveform; and
a function for calculating, based on the first waveform information and the second waveform information, a pulse wave transit time, which is a difference between a timing corresponding to a first feature which is a characteristic feature of the first waveform and the timing corresponding to the second feature which is a characteristic feature of the second waveform.
